(19) United States
(12) Patent Application Publication
(10) Pub. No.: US 2015/0216795 A1
(43) Pub. Date: Aug. 6, 2015

(54) ARTICLE OF MANUFACTURE COMPRISING AFLIBERCEPT OR ZIV-AFLIBERCEPT

(71) Applicant: SANOFI, PARIS (FR)

(72) Inventors: Sylvie ASSADOURIAN, Paris (FR); Rémi CASTAN, Paris (FR); Emmanuelle MAGHERINI, Paris (FR)

(21) Appl. No.: 14/611,561

(22) Filed: Feb. 2, 2015

Related U.S. Application Data
(63) Continuation of application No. PCT/EP2013/066299, filed on Aug. 2, 2013.
(60) Provisional application No. 61/678,983, filed on Aug. 2, 2012.
(60) Provisional application No. 61/679,490, filed on Aug. 3, 2012.

(51) Int. Cl. A61K 9/00; B65D 85/00; A61K 31/4745; A61K 31/513; A61K 38/17; A61K 31/519

(52) U.S. Cl. CPC A61K 9/0019; A61K 38/179; A61K 31/519; A61K 31/4745; A61K 31/513; B65D 85/70

(57) ABSTRACT

Article of manufacture comprising a packaging material, a polypeptide of SEQ ID NO:1, aflibercept or ziv-aflibercept or a biosimilar thereof, and a label comprising a printed statement which informs a prospective user of adverse events or adverse reactions.

### REFERENCE TO RELATED APPLICATIONS

This application is a continuation of International Patent Application No. PCT/EP2013/066299, filed Aug. 2, 2013, which claims the benefit of U.S. Provisional Application Ser. No. 61/678,983, filed on Aug. 2, 2012 and Ser. No. 61/679,490, filed on Aug. 3, 2012 which are herein incorporated by reference.

### FIELD

The present invention relates to an article of manufacture comprising a polypeptide of SEQ ID NO:1, or a biosimilar thereof, a packaging material, and a label comprising a printed statement which informs a prospective user of adverse events or adverse reactions. In one embodiment, polypeptide is aflibercept. In another embodiment, the polypeptide is ziv-aflibercept or ZALTRAP®.

### BACKGROUND

Colorectal cancers are among the most frequent tumor types in the western countries, second to breast in women and third to lung and prostate in males. The end prognosis is dependent upon the extent of the disease. The five year survival rate in early localized stage of about 90%, decreased to approximately 60-65% after spread to adjacent organ(s) or lymph nodes and is of less than 10% after spread to distant sites.

When diagnosed before nodal involvement treatment is usually limited to surgical resection (and radiotherapy for patients with rectal cancer) and potential participation to clinical trials for adjuvant therapy. Patients with nodal involvement are candidates for adjuvant chemotherapy following initial surgery in the attempt to prevent metastatic recurrence of the disease. Once spread to distant sites treatment essentially consists of palliative chemotherapy.

About 75 to 80% of all the patients with colorectal carcinoma will present at a stage when all gross carcinoma can be surgically removed. However, almost half of these patients will ultimately die from metastatic disease. Furthermore 20 to 25% of the patients present with metastatic disease at diagnosis. Once metastases are present median overall survival with available combination therapy is around 20 months.

Over the past decades 5-Fluorouracil (5-FU) has remained the mainstay of the chemotherapy in colorectal cancer. During years the major determinant in the treatment of colorectal cancer patients has been the improvement in the schedules of 5-FU administration.

Among these, the bimonthly regimen (LV5FU2) of 5-FU given as bolus/infusion over 2 days has been shown to be superior to the monthly 5 day bolus regimen (Mayo regimen) in terms of response rate (RR) (32.6% vs 14.4%), in terms of progression free survival (PFS) (27.6 vs 22.0 weeks), and safety (de Gramont et al, Journal of Clinical Oncology 1997; 15(2):808-815).

However, no statistically significant improvement in the overall survival (OS) was seen until development, starting in the beginning of nineties, of two novel cytotoxic agents, oxaliplatin, a DACH platinum, and the topoisomerase I inhibitor, irinotecan. With each of these two new agents median overall survival in the first line metastatic setting reached 15 to 19 months in multiple Phase III trials.

In a study, published in 2004 by Tournigand et al. (Journal of Clinical Oncology 2004; 22(2):229-237), where these two drugs were administered in sequence in the same protocol, as first then second line treatment in metastatic colorectal cancer patients, the threshold of 20 months median overall survival was crossed whatever was the order of the treatment sequence.

Recently, new therapeutic approaches have emerged. One such therapy involves the biologic aflibercept. The protein, also referred to as VEGFR1R2-Fc.DELTA.C1 or Flt1D2.F1k1D3.Fc.DELTA.C1, is a homo dimer, with each dimer comprising two identical monomers, each of which is a fusion protein comprising the signal sequence of VEGFR1 fused to the D2 Ig domain of the VEGFR1 receptor, itself fused to the D3 Ig domain of the VEGFR2 receptor, in turn fused to the Fc domain of IgG1.

The protein chain is glycosylated, with N-acetyl-glucosamine, fucose, galactose, mannose and sialic acids contributing to the carbohydrate structures. The N-linked oligosaccharides consist of mainly bi-antennary structures with zero, one or two terminal sialic acids. The amino acid sequence (SEQ ID NO1) of the monomer is provided on FIG. 1.

The U.S. Food and Drug Administration (FDA) approved aflibercept under the trade name EYLEA® for the treatment of patients with neovascular (wet) age-related macular degeneration (AMD). In particular, EYLEA® is the trade name for aflibercept as generated, processed and formulated for intravitreal injection.

In light of aflibercept's approved use in treating AMD, the FDA requested that a different name (ziv-aflibercept) be given for the compound's use in the treatment of cancer. Thus, ziv-aflibercept is the United States Adopted Name (USAN) accepted by FDA to designate a pharmaceutical composition comprising aflibercept as generated, processed and formulated for injection via intravenous infusion. Ziv-aflibercept has been approved by the FDA for sale under the tradename ZALTRAP® for the treatment of metastatic colorectal cancer (mCRC).

The European Medicines Agency (EMA) did not request separate names for the compound. Thus, in the European Union the name “aflibercept” is used regardless of the indication.

ZALTRAP® and EYLEA® are obtained by slightly different processes. They both contain aflibercept or ziv-aflibercept, but the ratio of aggregates of aflibercept or ziv-aflibercept is slightly different in ZALTRAP® and EYLEA®.

In a phase I study (TCD6118), aflibercept was administered IV in combination with irinotecan (180 mg/m² on day 1), leucovorin (200 mg/m² on day 1 and day 2), and 5-FU (bolus/infusional 400/600 mg/m² on day 1 and day 2), every 2 weeks in patients with advanced solid malignancies. The aflibercept 4 mg/kg dose every 2 weeks was considered to be the optimum dose.

In a phase II trial (NCI7498), aflibercept was administered in previously treated patients with metastatic colorectal cancer. This trial showed that aflibercept is well tolerated in pre-treated patients with MCRC. The trials suggested that aflibercept as single agent or in combination should be explored (Tang et al, J Clin Oncol 26: 2008 (May 20 suppl; abstr 4027).

But the results provided in these two studies provided no insight as to efficacy.

Furthermore, a phase III trial of aflibercept in metastatic pancreatic cancer was discontinued in 2009, and in 2011 the data of a phase III trial evaluating aflibercept for the second-line treatment of non-small cell lung cancer (NSCLC) showed that adding aflibercept to the chemotherapy drug docetaxel did not meet the pre-specified criteria for the primary endpoint of improvement in overall survival compared with a regimen of docetaxel plus placebo.

It has been recently discovered that aflibercept or ziv-aflibercept may have significant effects on patients suffering from Colorectal Cancer (CRC) and in particular metastatic Colorectal Cancer (CRC).

During clinical trials with ziv-aflibercept in patients suffering from Colorectal Cancer (CRC) and in particular metastatic Colorectal Cancer (CRC), some adverse events or adverse reactions of ziv-aflibercept were reported. Accordingly, improved methods and articles are needed to manage the risk of adverse events arising from the use of aflibercept in the treatment of patients with colorectal cancer.

### SUMMARY

Therefore the treatment of Colorectal Cancer (CRC) and in particular metastatic Colorectal Cancer (CRC) with ziv-aflibercept or aflibercept is contra-indicated for patients as indicated below:
i) patients with a with severe haemorrhage, or
ii) patients who experience gastrointestinal perforation, or
iii) patients with compromised wound healing.

During clinical trials further adverse events or adverse reactions have been reported:
Fistula Formation
Hypertension: Monitor blood pressure and treat hypertension.
Arterial Thromboembolic Events (ATE) (e.g., transient ischemic attacks, cerebrovascular accident, angina pectoris):
Proteinuria: Monitor urine protein
Neutropenia and Neutropenic Complications:
Diarrhea and Dehydration: Incidence of severe diarrhea and dehydration is increased.
Reversible Posterior Leukoencephalopathy Syndrome (RPLS)

Depending on the grade of these further adverse events or adverse reactions the administration aflibercept or ziv-aflibercept should be discontinued, temporally suspended or delayed.

Therefore the treatment of patients suffering from Colorectal Cancer (CRC) and in particular metastatic Colorectal Cancer (CRC) with aflibercept or ziv-aflibercept has to be managed to avoid death.

The Applicant has now found methods for managing the risk related to aflibercept or ziv-aflibercept.

The methods according to the invention enable to decrease the risk of said events, when aflibercept or ziv-aflibercept is administered for treating Colorectal Cancer (CRC) and in particular metastatic Colorectal Cancer (CRC).

The invention relates to methods, compositions and articles as disclosed herein.

In one aspect the invention provides for an article of manufacture comprising:

a) a packaging material
b) aflibercept or ziv-aflibercept or ZALTRAP®, and
c) a label or package insert contained within said packaging, said label comprising a printed statement which informs a prospective user that:
i) aflibercept or ziv-aflibercept or ZALTRAP® should not be administered to patients with a with severe haemorrhage, or
ii) aflibercept or ziv-aflibercept or ZALTRAP® therapy should be discontinued in patients who experience gastrointestinal perforation, or
iii) aflibercept or ziv-aflibercept or ZALTRAP® therapy should be discontinued in patients with compromised wound healing.

In an embodiment the label or package insert contained within said packaging material further indicates that aflibercept or ziv-aflibercept or ZALTRAP® in combination with folinic acid, 5-fluorouracil (5-FU) and irinotecan is indicated for patients with Colorectal Cancer (CRC) or Colorectal Cancer (CRC) symptom.

In a further embodiment the label or package insert contained within said packaging material further indicates that aflibercept or ziv-aflibercept or ZALTRAP® in combination with folinic acid, 5-fluorouracil (5-FU) and irinotecan is indicated for patients with Metastatic Colorectal Cancer (mCRC).

In a further embodiment the label or package insert contained within said packaging material further indicates that aflibercept or ziv-aflibercept or ZALTRAP® in combination with folinic acid, 5-fluorouracil (5-FU) and irinotecan is indicated for patients with metastatic colorectal cancer (mCRC) that is resistant to or has progressed following an oxaliplatin-containing regimen.

In a further embodiment the article of manufacture comprises in separate containers pharmaceutical compositions for combined use in treating CRC in a patient which comprises (1) a pharmaceutical composition comprising aflibercept or viz-aflibercept, (2) a pharmaceutical composition comprising folinic acid, (3) a pharmaceutical composition comprising 5-fluorouracil (5-FU) and (4) a pharmaceutical composition comprising irinocetan.

In a further embodiment, the pharmaceutical composition comprising the polypeptide aflibercept or viz-aflibercept is hyperosmolar.

In a further embodiment, the osmolarity of the pharmaceutical composition comprising the polypeptide aflibercept or viz-aflibercept is higher than 500 mOsm, 700 mOsm, 900 mOsm or 1000 mOsm.

In a further embodiment the aflibercept or viz-aflibercept or the polypeptide of SEQ ID NO:1, or a biosimilar thereof is sialilated In a further embodiment the aflibercept or viz-aflibercept or the polypeptide of SEQ ID NO:1, or a biosimilar thereof contains about 8 to about 12 moles of sialic acid/moles of polypeptide.

In a further embodiment the polypeptide of SEQ ID NO:1, or a biosimilar thereof forms a dimer.

In a further embodiment the label or package insert contained within said packaging material further indicates that 4 mg/kg of aflibercept or ziv-aflibercept or ZALTRAP® are administered as an intravenous infusion over 1 hour every 2 weeks.

In a further embodiment the label or package insert contained within said packaging material further indicates that aflibercept or ziv-aflibercept or ZALTRAP® should not be administered as an intravenous (IV) push or bolus

In a further embodiment the article of manufacture comprises single-use vials containing 100 mg/4 mL of aflibercept or ziv-aflibercept or ZALTRAP® (25 mg/mL) or 200 mg/8 mL of aflibercept or ziv-aflibercept or ZALTRAP® (25 mg/mL).

In a further embodiment the article of manufacture wherein said label or package insert comprises a printed statement comprising the following information:

### WARNING: HEMORRHAGE, GASTROINTESTINAL PERFORATION, COMPROMISED WOUND HEALING

Hemorrhage: Severe and sometimes fatal hemorrhage, including gastrointestinal (GI) hemorrhage, has been reported in the patients who have received ZALTRAP in combination with FOLFIRI. Monitor patients for signs and symptoms of GI bleeding and other severe bleeding. Do not administer ZALTRAP to patients with severe hemorrhage [see Dosage and Administration (2.2), Warnings and Precautions (5.1)].
Gastrointestinal Perforation: Gastrointestinal (GI) perforation including fatal GI perforation can occur in patients receiving ZALTRAP. Discontinue ZALTRAP therapy in patients who experience GI perforation [see Dosage and Administration (2.2), Warnings and Precautions (5.2)].

Compromised Wound Healing: Severe compromised wound healing can occur in patients receiving ZALTRAP/FOLFIRL Discontinue ZALTRAP in patients with compromised wound healing. Suspend ZALTRAP for at least 4 weeks prior to elective surgery, and do not resume ZALTRAP for at least 4 weeks following major surgery and until the surgical wound is fully healed [see Dosage and Administration (2.2), Warnings and Precautions (5.3)].

In a further aspect, the invention relates to a method of treating Colorectal Cancer (CRC) or Colorectal Cancer (CRC) symptom in a patient in need thereof comprises administering to said patient therapeutically effective amounts of aflibercept or ziv-aflibercept or ZALTRAP®, folinic acid, 5-fluorouracil (5-FU) and irinotecan wherein:
iv) aflibercept or ziv-aflibercept or ZALTRAP® should not be administered to patients with a with severe haemorrhage, or
v) aflibercept or ziv-aflibercept or ZALTRAP® therapy should be discontinued in patients who experience gastrointestinal perforation, or
vi) aflibercept or ziv-aflibercept or ZALTRAP® therapy should be discontinued in patients with compromised wound healing.

In an embodiment of the method, said patient has already been treated for the CRC or CRC symptom (second-line treatment).

In a further embodiment, CRC is a Metastatic Colorectal Cancer.

However, the method can be used for treating any other cancer such as lung cancer, gastric cancer, ovarian cancer and any other cancer susceptible to be treated with aflibercept.

The method can be used for treating cancer with aflibercept as single agent or with aflibercept in combination with other chemical molecule or biologic molecule of the cancer. Such biologic molecule can be for instance an anti-Ang2 antibody.

In a further embodiment, said patient has previously been treated with chemotherapy, radiotherapy or surgery. In one embodiment, said patient has failed chemotherapy, radiotherapy or surgery.

In a further embodiment, said patient has previously been treated with therapy based on oxaliplatin or on bevacizumab.

In a further embodiment, said patient has failed therapy based on oxaliplatin or on bevacizumab.

In a further embodiment, the invention provides a method wherein folinic acid at a dosage comprised between about 200 mg/m² and about 600 mg/m², 5-fluorouracil (5-FU) at a dosage comprised between about 2000 mg/m² and about 4000 mg/m², irinotecan at a dosage comprised between about 100 mg/m² and about 300 mg/m² and aflibercept at a dosage comprised between about 1 mg/kg and about 10 mg/kg are administered to patient.

In the present application, the dosage of folinic acid indicated should be understood as the dosage of the racemate of folinic acid, i.e. comprising the D and L forms. Should only the L form be used the dosage should be half of the dosage indicated for the racemate.

In other words a dosage of folinic acid of about 200 mg/m² as indicated in the present application corresponds to about 200 mg/m² of racemate and about 100 mg/m² of L form.

In a further embodiment, the invention provides a method wherein folinic acid at a dosage of about 400 mg/m², 5-fluorouracil (5-FU) at a dosage of about 2800 mg/m², irinotecan at a dosage of about 180 mg/m² and aflibercept at a dosage of about 4 mg/kg are administered to patient.

In a further embodiment, the invention provides a method wherein said patient receives intravenous folinic acid at a dosage comprised of about 400 mg/m², intravenous 5-fluorouracil (5-FU) at a dosage of about 2800 mg/m², intravenous irinotecan at a dosage comprised of about 180 mg/m² and intravenous aflibercept at a dosage of about 4 mg/kg every two weeks.

In a further embodiment, the invention provides a method wherein said patient receives intravenous folinic acid, intravenous 5-fluorouracil (5-FU), intravenous irinotecan and intravenous aflibercept every two weeks for a period comprised between about 9 and about 18 weeks.

In a further embodiment, the invention provides a method wherein said patient receives intravenous folinic acid immediately after aflibercept administration.

In a further embodiment, the invention provides a method wherein said patient receives intravenous irinotecan immediately after aflibercept administration.

In a further embodiment, the invention provides a method wherein said patient receives intravenous irinotecan immediately after aflibercept administration over almost 90 minutes.

In a further embodiment, the invention provides a method wherein said patient receives intravenous 5-fluorouracil (5-FU) immediately after aflibercept administration.

In a further embodiment, the invention provides a method wherein said patient receives a first quantity of intravenous 5-fluorouracil (5-FU) immediately after aflibercept administration and a second quantity in continuos infusion.

In a further embodiment the invention provides a method wherein said patient receives about 400 mg/m² of intravenous 5-fluorouracil (5-FU) over about 2 to 4 minutes after aflibercept administration and 2400 mg/m² over about 46 hours after aflibercept administration in continuous infusion.

In a further embodiment aflibercept or ziv-aflibercept or ZALTRAP is administered at 4 mg per kg as an intravenous (IV) infusion over 1 hour every two weeks. In a further embodiment aflibercept or ziv-aflibercept or ZALTRAP is administered prior to any component of the FOLFIRI regimen on the day of treatment.

Aflibercept or ziv-aflibercept or ZALTRAP treatment is continued until disease progression or unacceptable toxicity.

In a further embodiment the patient has liver metastases.

In a further aspect, the invention relates to a method of promoting the use of a polypeptide of SEQ ID NO:1, or a biosimilar thereof, the method comprising the step of conveying to a recipient at least one message selected from the group consisting of:
a. the polypeptide, or biosimilar thereof, should not be administered to patients with severe haemorrhage, and/or
b. the polypeptide, or biosimilar thereof, should be discontinued in patients who experience gastrointestinal perforation, and/or
c. the polypeptide, or biosimilar thereof, should be discontinued in patients with compromised wound healing.

In a further aspect, the invention relates to a method of managing the risk of hemorrhage, gastrointestinal perforation and compromised wound healing to allow a safe and effective use of a regiment comprising a polypeptide of SEQ ID NO:1, or biosimilar thereof, leucovorin, 5-fluorouracil (5-FU) and irinotecan in the treatment of patients with colorectal cancer (CRC), said method comprising,

a) assessing whether a patient presents severe haemorrhage, and, if not, administering to said patient said regiment;
b) monitoring said patient for signs of gastrointestional perforation or compromised wound healing; and
c) discontinuing the regiment if said signs appear.

In one aspect, there is provided a polypeptide of SEQ ID NO:1, or biosimilar thereof, for use in treating patients with cancer or cancer symptom wherein:
a. the polypeptide, or biosimilar thereof, should not be administered to patients with a with severe haemorrhage, and/or
b. the polypeptide, or biosimilar thereof, should be discontinued in patients who experience gastrointestinal perforation, and/or
c. the polypeptide, or biosimilar thereof, should be discontinued in patients with compromised wound healing.

In another aspect, a composition is provided comprising therapeutically effective amounts of a polypeptide of SEQ ID NO:1, or biosimilar thereof, in combination with folinic acid, 5-fluorouracil (5-FU) and irinocetan and comprising a pharmaceutically acceptable carrier for use in treating patients with Colorectal Cancer (CRC) or Colorectal Cancer (CRC) symptom wherein:
a. the polypeptide, or biosimilar thereof, should not be administered to patients with a with severe haemorrhage, and/or
b. the polypeptide, or biosimilar thereof, should be discontinued in patients who experience gastrointestinal perforation, and/or
c. the polypeptide, or biosimilar thereof, should be discontinued in patients with compromised wound healing.

The production process of aflibercept is typical for a recombinant-Fc fusion protein. The upstream process includes expansion of the CHO host cells and expression of recombinant aflibercept. The downstream process involves clarification and purification of the protein from the culture medium.

The manufacturing process is initiated with the thawing and inoculation of one working cell bank (WCB) vial. The cell culture is expanded until reaching sufficient density for inoculation into the production bioreactor.

The downstream process consists of several chromatography steps (protein A affinity chromatography, Cation exchange chromatography, Anion exchange chromatography and Hydrophobic Interaction chromatography), and includes viral inactivation and filtration steps to clear potential adventitious viral agents. After processing through the step of concentration/diafiltration, the drug substance is filled into containers and stored frozen.

Aflibercept can be formulated as described in WO2006/104852.

In particular Zaltrap is formulated in vials of 4 ml containing 100 mg of aflibercept (25 mg/ml) or in vials of 8 ml containing 200 mg of aflibercept (25 mg/ml).

Zaltrap formulation may also contain sucrose, sodium chloride, sodium citrate dihydrate, citric acid monohydrate, polysorbate 20, sodium phosphate dibasic heptahydrate, sodium phosphate monobasic monohydrate, sodium hydroxide and/or hydrochloric acid and water for injections.

However aflibercept can be formulated differently if it is intended to be administered by other ways for instance by sub cutaneous administration.

If the sub cutaneous administration route is used aflibercept can be formulated as described in U.S. Pat. No. 8,404,638.

The man skilled in the art may refer to carry out the present invention in particular to WO2006/104852 or to WO 00/75319 which are incorporated by reference.

### BRIEF DESCRIPTION OF THE DRAWINGS

FIG. 1—Aflibercept amino acid sequence (SEQ ID NO:1)

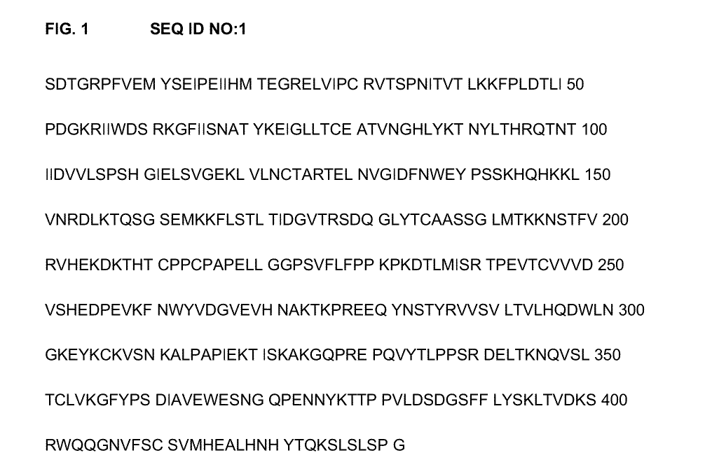

FIG. 2—Overall survival (months)—Kaplan-Meier curves by treatment group—ITT population

FIG. 3—Overall survival (months)—Subgroup analyses (forest plot)—By stratification factors as per IVRS—ITT population

FIG. 4—Overall survival (months)—Subgroup analyses (forest plot)—By patient demographics—ITT population

FIG. 5—Overall survival (months)—Subgroup analyses (forest plot)—By baseline characteristics—ITT population

FIG. 6—PFS based on tumor assessment by the IRC (months)—Subgroup analysis (forest plot)—By stratification factors as per IVRS—ITT population

### DETAILED DESCRIPTION

### Definitions

5-fluorouracil (5-FU or f5U) is a pyrimidine analog used in the treatment of cancer. It is a suicide inhibitor and works through irreversible inhibition of thymidylate synthase. It belongs to the family of drugs called antimetabolites.

Folinic acid, also called leucovorin, is an adjuvant to cancer chemotherapy used in combination with 5-fluorouracil.

Irinotecan is a drug used for the treatment of cancer. Irinotecan is a topoisomerase 1 inhibitor, which prevents DNA from unwinding.

FOLFIRI is the combination of folinic acid, 5-fluorouracil (5-FU) and irinotecan and will be used throughout the document.

As used herein, the term “biosimilar” shall refer to a product approved for the treatment of a disease indication (e.g., cancer) under 42 U.S.C. 262(k), or under article 10(4) of Directive 2001/83/EC or under an equivalent statute of another jurisdiction. In one embodiment, the biosimilar is approved for the treatment of colorectal cancer. In another embodiment, the biosimilar is approved with a label comprising the information in Example 2 infra as a printed statement.

The present invention relates to any biosimilar of aflibercept or ziv-aflibercept, whatever is the name given in the registers (such as INN or USAN name), as far it has the sequence SEQ ID NO:1, or consists of dimer of this sequence or of a sequence having at least 97% identity with SEQ ID NO:1.

### Example 1

### EFC10262 (VELOUR)/a Multinational, Randomized, Double-Blind Study, Comparing the Efficacy of Ziv-Aflibercept Once Every 2 Weeks Versus Placebo in Patients with Metastatic Colorectal Cancer (MCRC) Treated with Irinotecan/5-FU Combination (FOLFIRI) after Failure of an Oxaliplatin Based Regimen

EFC10262 was designed as a randomized, double-blind, multi-centre study comparing ziv-aflibercept at 4 mg/kg to placebo, in combination with Irinotecan and 5 Fluorouracil combination (FOLFIRI) given intravenously every 2 weeks as second line treatment for patients with metastatic colorectal cancer (MCRC) after failure of an oxaliplatin based regimen. Each randomized patient was to be treated until disease progression, death, or unacceptable toxicity. The primary objective of EFC10262 was to demonstrate improvement in overall survival (OS) for ziv-aflibercept+FOLFIRI compared to placebo+FOLFIRI. The predefined statistical significance level for this final analysis was 0.0466 after adjusting the type I error spent for the two interim analyses using the O'Brien-Fleming spending function.

The study included one formal interim analysis, planned for the purpose of efficacy, when 561 death events (65% information time) had occurred. Upon request of the independent Data Monitoring Committee (DMC), an additional interim analysis of OS was performed to provide an early evaluation of the benefit-risk ratio, when 315 death events (36.5% information fraction) had occurred.

A total of approximately 863 deaths were required to detect 20% hazard rate reduction in OS with 90% power using the two-sided log rank test at an overall 0.0499 alpha level. The median survival times was expected to be 11 months for the control group. The overall alpha level was split between overall survival (0.0499) and progression-free survival as a secondary efficacy endpoint (0.0001).

Approximately 1200 patients (i.e. 600 patients per treatment group) were planned to be randomized. Treatment assignment was stratified according to prior therapy with bevacizumab (yes or no), and ECOG performance status (PS) (0 vs 1 vs 2).

The enrolment started in November 2007 and was completed in March 2010. A total of 1226 patients were randomized. The efficacy analysis was based on all randomized patients (Intent-to-Treat (ITT) population: 614 in the placebo arm and 612 patients in the ziv-aflibercept arm). The safety analysis was based on all treated patients (safety population: 605 and 611 patients in the placebo and ziv-aflibercept arms, respectively). Treatment arms were evenly balanced for demographics, disease characteristics and prior anti-cancer treatments, including prior exposition to bevacizumab.

### Dosage and Schedule of Administration

Patients were administered either ziv-aflibercept or placebo, depending on arm assigned. Immediately after, patients received irinotecan, 5-FU and leucovorin (FOLFIRI regimen). This treatment was repeated every 2 weeks.

### Ziv-Aflibercept/Placebo

Arm A, ziv-aflibercept: 4 mg/kg was administered IV over 1 hour on Day 1, every 2 weeks,

OR

Arm B, placebo: 4 mg/kg was administered IV over 1 hour on Day 1, every 2 weeks.

### FOLFIRI Regimen

Immediately after ziv-aflibercept/placebo administration, all the patients received:
Irinotecan 180 mg/m² IV infusion in 500 mL in 5% dextrose solution in water (D5W) over 90 minutes and dextro-levogyre (dl) leucovorin 400 mg/m² IV infusion over 2 hours, at the same time, in bags using a Y-line, followed by:
5-FU 400 mg/m² IV bolus given over 2-4 minutes, followed by:
5-FU 2400 mg/m² continuous IV infusion in 500 mL D5W (recommended) over 46-hours.

### Results of EFC10262

Demographics and Baseline Characteristics Patient demographics and characteristics at baseline were similar the 2 treatment arms (Table 1).

TABLE 1
Summary of patient demographics and patient
characteristics at baseline - ITT population

| | | Ziv-aflibercept/ | |
|---|---|---|---|
| | Placebo/Folfiri | Folfiri | All |
| | (N = 614) | (N = 612) | (N = 1226) |
| Gender [n(%)] | | | |
| Number | 614 | 612 | 1226 |
| Male | 353 (57.5%) | 365 (59.6%) | 718 (58.6%) |
| Female | 261 (42.5%) | 247 (40.4%) | 508 (41.4%) |
| Age (Years) | | | |
| Number | 614 | 612 | 1226 |
| Median | 61.0 | 61.0 | 61.0 |
| Mean (SD) | 60.2 (10.8) | 59.5 (10.5) | 59.8 (10.7) |
| Min:Max | 19:86 | 21:82 | 19:86 |
| Age class [n(%)] | | | |
| Number | 614 | 612 | 1226 |
| <65 | 376 (61.2%) | 407 (66.5%) | 783 (63.9%) |
| ≧65 but <75 | 199 (32.4%) | 172 (28.1%) | 371 (30.3%) |
| ≧75 | 39 (6.4%) | 33 (5.4%) | 72 (5.9%) |
| Race [n(%)] | | | |
| Number | 614 | 612 | 1226 |
| Caucasian/White | 523 (85.2%) | 548 (89.5%) | 1071 (87.4%) |
| Black | 27 (4.4%) | 16 (2.6%) | 43 (3.5%) |
| Asian/Oriental | 51 (8.3%) | 35 (5.7%) | 86 (7.0%) |
| Other | 13 (2.1%) | 13 (2.1%) | 26 (2.1%) |
| Region | | | |
| Number | 614 | 612 | 1226 |
| Western Europe | 217 (35.3%) | 208 (34.0%) | 425 (34.7%) |
| Eastern Europe | 136 (22.1%) | 161 (26.3%) | 297 (24.2%) |
| North America | 75 (12.2%) | 63 (10.3%) | 138 (11.3%) |
| South America | 56 (9.1%) | 62 (10.1%) | 118 (9.6%) |
| Other countries | 130 (21.2%) | 118 (19.3%) | 248 (20.2%) |

Note:
Other countries = Australia, New Zeland, South Africa and Korea

Disease characteristics at initial diagnosis and time from diagnosis to randomization were similar in the 2 treatment arms (Table 2).

TABLE 2
Disease characteristics at initial diagnosis - ITT population

| | | | Ziv-aflibercept/ | | | |
|---|---|---|---|---|---|---|
| | Placebo/Folfiri | | Folfiri | | All | |
| | (N = 614) | | (N = 612) | | (N = 1226) | |
| Primary site | | | | | | |
| [n(%)] | | | | | | |
| Number | 614 | | 612 | | 1226 | |
| Colon | 302 | (49.2%) | 289 | (47.2%) | 591 | (48.2%) |
| Recto sigmoid | 136 | (22.1%) | 123 | (20.1%) | 259 | (21.1%) |
| Rectum | 174 | (28.3%) | 197 | (32.2%) | 371 | (30.3%) |
| Other | 2 | (0.3%) | 3 | (0.5%) | 5 | (0.4%) |
| cea & ck20 postive - | 1 | (0.2%) | 0 | | 1 | (<0.1%) |
| presumed colorectal | | | | | | |
| primary | | | | | | |
| Appendix | 0 | | 1 | (0.2%) | 1 | (<0.1%) |
| Colon plus appendix | 0 | | 1 | (0.2%) | 1 | (<0.1%) |
| Presumed colorectal, | 0 | | 1 | (0.2%) | 1 | (<0.1%) |
| cea positive and | | | | | | |
| history of colon | | | | | | |
| cancer >20 years ago | | | | | | |
| Synchronous primary, | 1 | (0.2%) | 0 | | 1 | (<0.1%) |
| cecum and rectum | | | | | | |
| Histology type | | | | | | |
| [n(%)] | | | | | | |
| Number | 614 | | 612 | | 1226 | |
| Adenocarcinoma | 614 | (100%) | 612 | (100%) | 1226 | (100%) |
| Time from 1^{st} | | | | | | |
| diagnosis to | | | | | | |
| randomization | | | | | | |
| (months) [n(%)]* | | | | | | |
| Number | 614 | | 611 | | 1225 | |
| Mean (SD) | 20.88 | (21.10) | 20.98 | (24.08) | 20.93 | (22.62) |

| | | | |
|---|---|---|---|
| Median | 13.67 | 14.62 | 14.26 |
| Min:Max | 2.4:214.7 | 2.1:325.1 | 2.1:325.1 |

*If the day of initial date of diagnosis is missing, it is considered as the first day of the month

### Patient Accountability

Overall, 30.4% of the randomized patients were allocated in the prior bevacizumab stratum (Table 3).

TABLE 3
Summary of randomized patients by stratification
factor (as per IVRS) - ITT population

| | | Ziv-aflibercept/ | |
|---|---|---|---|
| | Placebo/Folfiri | Folfiri | All |
| Stratification factors | (N = 614) | (N = 612) | (N = 1226) |
| ECOG PS [n(%)] | | | |
| 0 | 350 (57.0%) | 349 (57.0%) | 699 (57.0%) |
| 1 | 250 (40.7%) | 250 (40.8%) | 500 (40.8%) |
| 2 | 14 (2.3%) | 13 (2.1%) | 27 (2.2%) |
| Prior Bevacizumab | | | |
| [n(%)] | | | |
| Yes | 187 (30.5%) | 186 (30.4%) | 373 (30.4%) |
| No | 427 (69.5%) | 426 (69.6%) | 853 (69.6%) |

Note:
ECOG: Eastern Cooperative Oncology Group,
PS: Performance Status,
IVRS: Interactive Voice response System

### Dosage and Duration

The median overall study treatment exposure (i.e. either both study drugs ziv-aflibercept/placebo and FOLFIRI, or one of them alone) was 8.0 and 9.0 cycles in the placebo and ziv-aflibercept treatment arms, respectively (Table 4).

TABLE 4
Summary of overall study treatment exposure - Safety population

| Number of cycles | Placebo/Folfiri | Ziv-aflibercept/Folfiri |
|---|---|---|
| received by patient | (N = 605) | (N = 611) |
| Sum | 6127 | 6358 |
| Mean (SD) | 10.1 (8.1) | 10.4 (7.6) |
| Median | 8.0 | 9.0 |
| Min:Max | 1:67 | 1:50 |

SD: standard deviation

The median number of ziv-aflibercept/placebo infusions was 8.0 and 7.0 in the placebo and ziv-aflibercept treatment arms, respectively (Table 5). The median relative dose intensity was 83% with ziv-aflibercept as compared to 92% with placebo.

TABLE 5
Exposure to Ziv-aflibercept/Placebo - Safety population

| | Placebo/Folfiri | | Ziv-aflibercept/Folfiri | |
|---|---|---|---|---|
| | (N = 605) | | (N = 611) | |
| Number of cycles | | | | |
| received by patient | | | | |
| Sum | 6035 | | 5632 | |
| Mean (SD) | 10.0 | (8.0) | 9.2 | (7.2) |
| Median | 8.0 | | 7.0 | |
| Min:Max | 1:67 | | 1:35 | |
| 1 | 24 | (4.0%) | 43 | (7.0%) |
| 2 | 32 | (5.3%) | 52 | (8.5%) |
| 3 | 85 | (14.0%) | 70 | (11.5%) |
| 4 | 31 | (5.1%) | 45 | (7.4%) |
| 5 | 32 | (5.3%) | 43 | (7.0%) |
| 6 | 45 | (7.4%) | 29 | (4.7%) |
| 7 | 29 | (4.8%) | 28 | (4.6%) |
| 8 | 34 | (5.6%) | 29 | (4.7%) |
| 9 | 45 | (7.4%) | 29 | (4.7%) |
| 10 | 21 | (3.5%) | 28 | (4.6%) |
| 11-15 | 112 | (18.5%) | 94 | (15.4%) |
| 16-20 | 57 | (9.4%) | 68 | (11.1%) |
| 21-25 | 28 | (4.6%) | 34 | (5.6%) |
| >25 | 30 | (5.0%) | 19 | (3.1%) |
| Duration of exposure to ziv- | | | | |
| aflibercept/placebo (weeks) | | | | |
| Number | 605 | | 611 | |
| Mean (SD) | 22.3 | (17.5) | 21.7 | (16.7) |
| Median | 18.0 | | 17.9 | |
| Min:Max | 2:135 | | 2:85 | |
| Total cumulative | | | | |
| dose received (mg/kg) | | | | |
| Number | 605 | | 611 | |
| Mean (SD) | 39.63 | (31.65) | 35.69 | (27.96) |
| Median | 32.00 | | 28.00 | |
| Min:Max | 0.6:266.4 | | 3.8:140.0 | |
| Actual dose | | | | |
| intensity (mg/kg/week) | | | | |
| Number | 605 | | 611 | |
| Mean (SD) | 1.78 | (0.25) | 1.55 | (0.44) |
| Median | 1.84 | | 1.66 | |
| Min:Max | 0.3:2.1 | | 0.1:2.1 | |
| Relative dose intensity | | | | |
| Number | 605 | | 611 | |
| Mean (SD) | 0.89 | (0.12) | 0.78 | (0.22) |

| | | |
|---|---|---|
| Median | 0.92 | 0.83 |
| Min:Max | 0.2:1.1 | 0.1:1.1 |

Number of cycles received: Number of cycles with at least one dose infusion of ziv-aflibercept/placebo.

The median number of irinotecan infusions was 8.0 and 9.0 in the placebo and ziv-aflibercept treatment arms, respectively (table 6). The median relative dose intensity was 84% in the ziv-aflibercept arm as compared to 91% in the placebo arm. Of note, two patients did not receive irinotecan; the dose was considered equal to 0 for the calculation of the cumulative dose, actual and relative dose intensity.

TABLE 6
Exposure to irinotecan- Safety population

| | Placebo/Folfiri | | Ziv-aflibercept/Folfiri | |
|---|---|---|---|---|
| | (N = 605) | | (N = 611) | |
| Number of cycles | | | | |
| received by patient | | | | |
| Sum | 5992 | | 6157 | |
| Mean (SD) | 9.9 | (7.8) | 10.1 | (7.4) |
| Median | 8.0 | | 9.0 | |
| Min:Max | 1:67 | | 1:50 | |
| 1 | 23 | (3.8%) | 34 | (5.6%) |
| 2 | 29 | (4.8%) | 39 | (6.4%) |
| 3 | 87 | (14.4%) | 64 | (10.5%) |
| 4 | 33 | (5.5%) | 36 | (5.9%) |
| 5 | 29 | (4.8%) | 37 | (6.1%) |
| 6 | 48 | (7.9%) | 31 | (5.1%) |
| 7 | 27 | (4.5%) | 27 | (4.4%) |
| 8 | 32 | (5.3%) | 29 | (4.8%) |
| 9 | 47 | (7.8%) | 29 | (4.8%) |
| 10 | 21 | (3.5%) | 38 | (6.2%) |
| 11-15 | 114 | (18.9%) | 111 | (18.2%) |
| 16-20 | 58 | (9.6%) | 78 | (12.8%) |
| 21-25 | 31 | (5.1%) | 35 | (5.7%) |
| >25 | 25 | (4.1%) | 22 | (3.6%) |
| Duration of exposure | | | | |
| to irinotecan (weeks) | | | | |
| Number | 604 | | 610 | |
| Mean (SD) | 22.2 | (17.2) | 23.5 | (16.9) |
| Median | 18.1 | | 21.0 | |
| Min:Max | 2:135 | | 2:105 | |
| Total cumulative | | | | |
| dose received (mg/m²) | | | | |
| Number | 605 | | 611 | |
| Mean (SD) | 1736.30 | (1355.52) | 1730.37 | (1273.76) |
| Median | 1440.00 | | 1472.50 | |
| Min:Max | 0.0:11948.1 | | 0.0:9046.1 | |
| Actual dose | | | | |
| intensity (mg/m²/week) | | | | |
| Number | 605 | | 611 | |
| Mean (SD) | 78.82 | (11.74) | 73.59 | (13.68) |
| Median | 82.08 | | 75.60 | |
| Min:Max | 0.0:95.0 | | 0.0:95.0 | |
| Relative dose | | | | |
| intensity | | | | |
| Number | 605 | | 611 | |
| Mean (SD) | 0.88 | (0.13) | 0.82 | (0.15) |

| | | |
|---|---|---|
| Median | 0.91 | 0.84 |
| Min:Max | 0.0:1.1 | 0.0:1.1 |

Number of cycles received: Number of cycles with at least one dose infusion of irinotecan.

The median number of 5-FU infusions was 8.0 and 9.0 in the placebo and ziv-aflibercept treatment arms, respectively (Table 7). The median relative dose intensity was 83% in the ziv-aflibercept arm as compared to 91% in the placebo arm. Of note, two patients did not receive 5-FU; the dose was considered equal to 0 for the calculation of the cumulative dose, actual and relative dose intensity.

TABLE 7
Exposure to 5-FU - Safety population

| | Placebo/Folfiri | | Ziv-aflibercept/Folfiri | |
|---|---|---|---|---|
| | (N = 605) | | (N = 611) | |
| Number of cycles | | | | |
| received by patient | | | | |
| Sum | 6030 | | 6155 | |
| Mean (SD) | 10.0 | (7.9) | 10.1 | (7.4) |
| Median | 8.0 | | 9.0 | |
| Min:Max | 1:67 | | 1:50 | |
| 1 | 22 | (3.6%) | 35 | (5.7%) |
| 2 | 28 | (4.6%) | 39 | (6.4%) |
| 3 | 88 | (14.6%) | 63 | (10.3%) |
| 4 | 33 | (5.5%) | 35 | (5.7%) |
| 5 | 28 | (4.6%) | 37 | (6.1%) |
| 6 | 48 | (8.0%) | 32 | (5.2%) |
| 7 | 27 | (4.5%) | 28 | (4.6%) |
| 8 | 33 | (5.5%) | 28 | (4.6%) |
| 9 | 47 | (7.8%) | 29 | (4.7%) |
| 10 | 20 | (3.3%) | 39 | (6.4%) |
| 11-15 | 114 | (18.9%) | 113 | (18.5%) |
| 16-20 | 59 | (9.8%) | 77 | (12.6%) |
| 21-25 | 28 | (4.6%) | 35 | (5.7%) |
| >25 | 28 | (4.6%) | 21 | (3.4%) |
| Duration of | | | | |
| exposure to 5-FU | | | | |
| (weeks) | | | | |
| Number | 603 | | 611 | |
| Mean (SD) | 22.4 | (17.5) | 23.5 | (16.9) |
| Median | 18.1 | | 21.0 | |
| Min:Max | 2:135 | | 2:105 | |
| Total cumulative | | | | |
| dose received | | | | |
| (mg/m²) | | | | |
| Number | 605 | | 611 | |
| Mean (SD) | 27142.02 | (21341.89) | 26644.81 | (19245.24) |
| Median | 22400.00 | | 22702.44 | |
| Min:Max | 0.0:185874.8 | | 409.0:126701.4 | |
| Actual dose | | | | |
| intensity | | | | |
| (mg/m²/week) | | | | |
| Number | 605 | | 611 | |
| Mean (SD) | 1227.42 | (190.51) | 1140.36 | (214.35) |
| Median | 1276.38 | | 1165.56 | |
| Min:Max | 0.0:1477.3 | | 177.0:1491.3 | |
| Relative dose | | | | |
| intensity | | | | |
| Number | 605 | | 611 | |
| Mean (SD) | 0.88 | (0.14) | 0.81 | (0.15) |

| | | |
|---|---|---|
| Median | 0.91 | 0.83 |
| Min:Max | 0.0:1.1 | 0.1:1.1 |

Number of cycles received: Number of cycles with at least one dose infusion of 5-FU.

### Results of EFC10262

### 1. Overall Survival

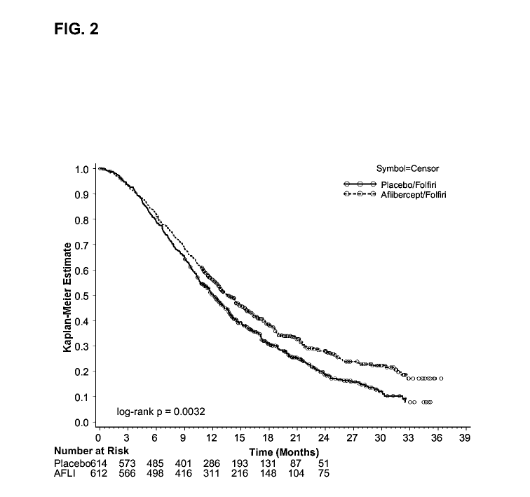

The median follow-up time at the cutoff date (7 Feb. 2011) for the ITT population was 22.28 months (FIG. 2 and Table 8). The study met its primary endpoint demonstrating a significant difference in overall survival in favor of ziv-aflibercept over placebo (stratified HR: 0.817, 95.34% Cl: 0.713 to 0.937; p=0.0032). The hazard ratio translates into a reduction of risk of death of 18.3% (95.34 Cl: 6.3% to 28.7%) with ziv-aflibercept compared to placebo. After 12 and 18 months from randomization, the estimated probabilities of being alive were 50.3% in placebo arm and 56.1% ziv-aflibercept arm, and 30.9% in placebo arm and 38.5% in ziv-aflibercept arm. Median overall survival was 13.50 months vs 12.06 months in ziv-aflibercept and placebo treatment arms, respectively. Sensitivity analyses and subgroup analyses showed a very consistent treatment effect confirming robustness of results on the primary endpoint.

TABLE 8
Overall survival (months) - Kaplan-Meier survival estimates by
treatment group- Primary analysis- Stratified according to stratification
factors at randomization (IVRS) - ITT population

| | Placebo/Folfiri | Ziv-aflibercept/Folfiri |
|---|---|---|
| Time to Event or Censoring | (N = 614) | (N = 612) |
| Overall | | |
| Number of death events, | 460/614 (74.9%) | 403/612 (65.8%) |
| n/N (%) | | |
| Median overall survival | 12.06 (11.072 | 13.50 (12.517 |
| (95.34% CI) (months) | to 13.109) | to 14.949) |
| Number of patients at risk | | |
| 3 months | 573 | 566 |
| 6 months | 485 | 498 |
| 9 months | 401 | 416 |
| 12 months | 286 | 311 |
| 18 months | 131 | 148 |
| 24 months | 51 | 75 |
| Survival probability | | |
| (95.34% CI) | | |
| 3 months | 0.935 (0.915 | 0.931 (0.911 |
| | to 0.955) | to 0.951) |
| 6 months | 0.791 (0.759 | 0.819 (0.788 |
| | to 0.824) | to 0.850) |
| 9 months | 0.654 (0.616 | 0.687 (0.650 |
| | to 0.692) | to 0.725) |
| 12 months | 0.503 (0.462 | 0.561 (0.521 |
| | to 0.543) | to 0.602) |
| 18 months | 0.309 (0.269 | 0.385 (0.343 |
| | to 0.348) | to 0.427) |
| 24 months | 0.187 (0.149 | 0.280 (0.237 |
| | to 0.225) | to 0.324) |
| Stratified Log-Rank | — | 0.0032 |
| test p-value^{a} | | |
| vs Placebo/Folfiri | | |
| Stratified Hazard ratio | — | 0.817 (0.713 |
| (95.34% CI)^{a} | | to 0.937) |
| vs Placebo/Folfiri | | |

Cutoff date = 7 FEB. 2011
Median follow-up time = 22.28 in months
^{a}Stratified on ECOG Performance Status (0 vs 1 vs 2) and Prior Bevacizumab (yes vs no) according to IVRS. Significance threshold is set to 0.0466 using the O'Brien-Fleming alpha spending function.

### Subgroup Analyses of Overall Survival (OS)

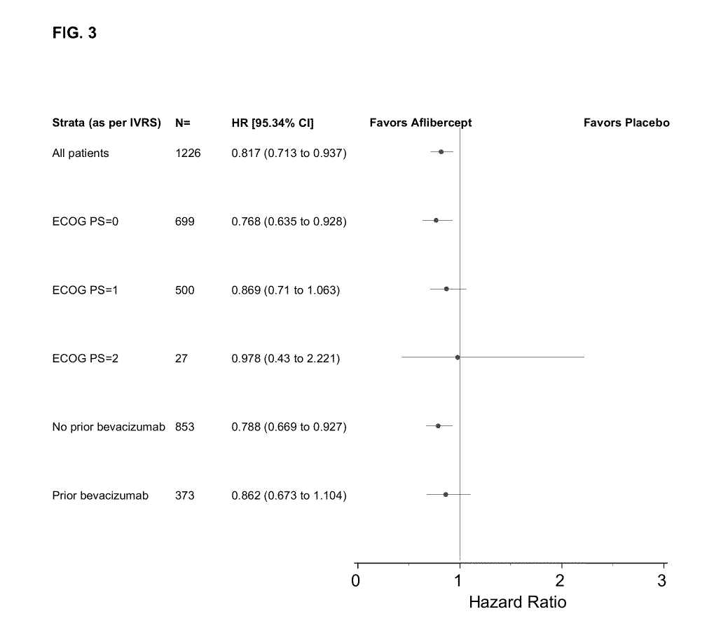

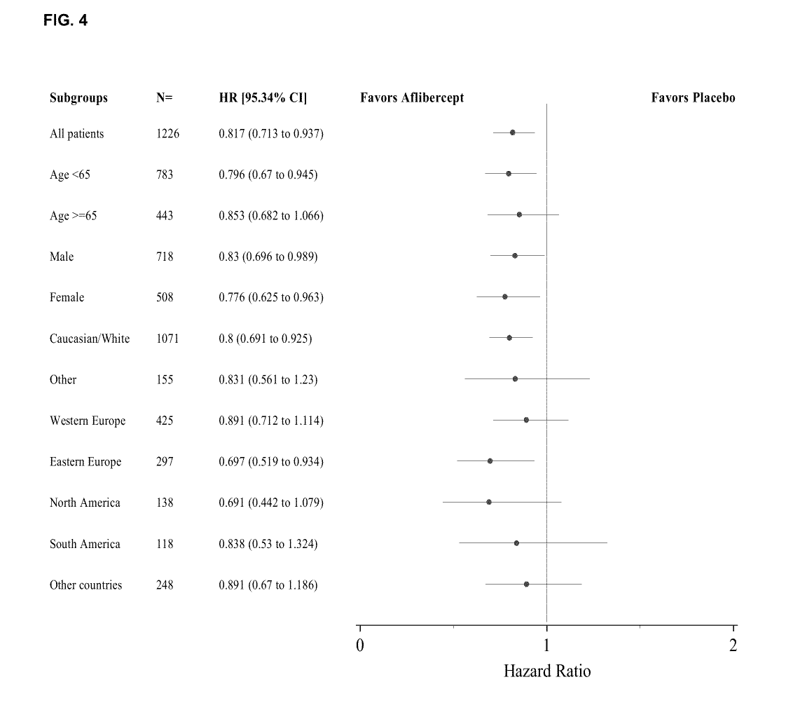

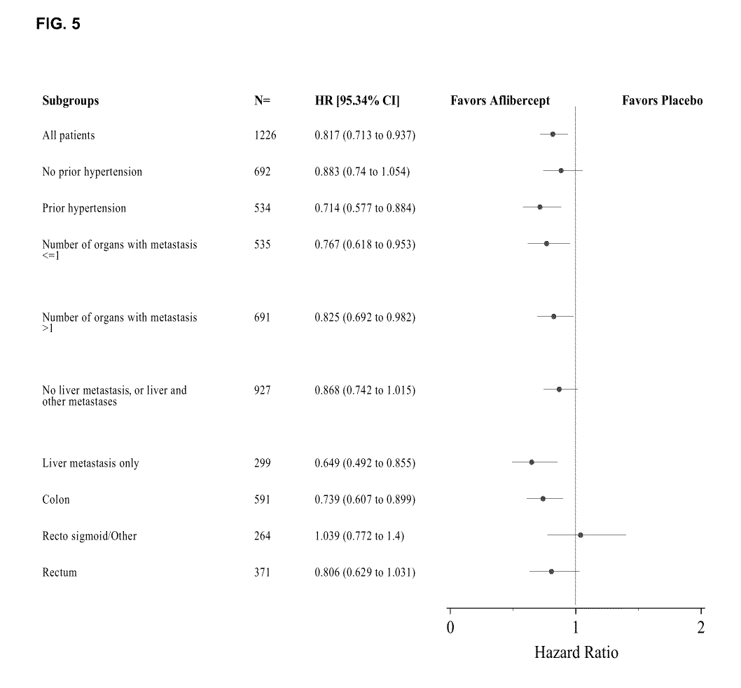

Subgroup analyses did not show any significant interaction (at the 2-sided 10% level) between treatment arms and stratification factors, indicating that the treatment effect was consistent across subgroups. This is illustrated in Table 9 and in FIGS. 3, 4 and 5.

TABLE 9
Overall survival (months) - Summary of subgroup analyses -
By stratification factors as per IVRS - ITT population

| | Placebo/Folfiri | Ziv-aflibercept/Folfiri | Hazard Ratio | |
|---|---|---|---|---|
| | Median (Months) | Median (Months) | (95.34% CI) vs | P-value for |
| | (95.34% CI) | (95.34% CI) | Placebo/Folfiri | interaction ^{a} |
| All patients | 12.1 (11.07 | 13.5 (12.52 | 0.817 (0.713 | |
| | to 13.11) | to 14.95) | to 0.937) | |
| Prior | | | | |
| bevacizumab | | | | |
| No | 12.4 (11.17 | 13.9 (12.71 | 0.788 (0.669 | 0.7231 |
| | to 13.54) | to 15.64) | to 0.927) | |
| Yes | 11.7 (9.82 | 12.5 (10.78 | 0.862 (0.673 | |
| | to 13.77) | to 15.51) | to 1.104) | |
| ECOG PS | | | | |
| 0 | 14.1 (12.88 | 16.9 (14.78 | 0.768 (0.635 | 0.5668 |
| | to 16.62) | to 18.79) | to 0.928) | |
| 1 | 10.1 (9.20 | 10.7 (9.36 | 0.869 (0.71 | |
| | to 11.53) | to 12.35) | to 1.063) | |
| 2 | 4.4 (1.97 | 2.8 (0.92 | 0.978 (0.43 | |
| | to 10.02) | to 9.82) | to 2.221) | |

Cutoff date = 7 FEB. 2011
Median follow-up time = 22.28 in months
^{a} Interaction test from the Cox proportional hazard model including the factor, treatment effect and the treatment by factor interaction

Treatment effect for OS was consistent across subgroups with regards to baseline characteristics at study entry. Of note, the interaction between treatment arms and the presence of liver metastasis factor was significant at 10% level, indicating a higher treatment effect in ‘liver metastasis only’ group (HR (95.34% Cl): 0.649 (0.492 to 0.855)) than in ‘no liver metastasis, or other metastases’ group (HR (95.34% Cl): 0.868 (0.742 to 1.015)) (quantitative interaction, p=0.0899) This is illustrated in Table 10.

TABLE 10
Overall survival (months) - Summary of subgroup analyses -
By baseline characteristics - ITT population

| | Placebo/Folfiri | Ziv-aflibercept/Folfiri | Hazard Ratio | |
|---|---|---|---|---|
| | Median (Months) | Median (Months) | (95.34% CI) vs | P-value for |
| | (95.34% CI) | (95.34% CI) | Placebo/Folfiri | interaction^{a} |
| All patients | 12.1 (11.07 | 13.5 (12.52 | 0.817 (0.713 | |
| | to 13.11) | to 14.95) | to 0.937) | |
| Prior | | | | |
| hypertension | | | | |
| No | 11.7 (10.41 | 12.7 (11.17 | 0.883 (0.74 | 0.1309 |
| | to 13.11) | to 14.39) | to 1.054) | |
| Yes | 12.7 (10.78 | 15.5 (12.91 | 0.714 (0.577 | |
| | to 14.00) | to 18.56) | to 0.884) | |
| Number of | | | | |
| metastatic organs | | | | |
| involved | | | | |
| >1 | 10.5 (9.72 | 12.1 (10.71 | 0.825 (0.692 | 0.6992 |
| | to 12.06) | to 13.11) | to 0.982) | |
| <=1 | 13.7 (12.29 | 16.0 (14.42 | 0.767 (0.618 | |
| | to 16.30) | to 20.86) | to 0.953) | |
| Liver Metastasis | | | | |
| only | | | | |
| No | 12.3 (11.07 | 13.2 (12.06 | 0.868 (0.742 | 0.0899 |
| | to 13.73) | to 15.28) | to 1.015) | |
| Yes | 11.4 (9.86 | 14.4 (12.68 | 0.649 (0.492 | |
| | to 12.88) | to 18.04) | to 0.855) | |
| Location of | | | | |
| primary tumor | | | | |
| Colon | 10.6 (9.66 | 12.9 (11.50 | 0.739 (0.607 | 0.1421 |
| | to 12.06) | to 16.16) | to 0.899) | |
| Recto | 14.1 (12.71 | 14.3 (12.35 | 1.039 (0.772 | |
| sigmoid/Other | to 17.08) | to 16.39) | to 1.4) | |
| Rectum | 12.6 (10.35 | 13.5 (11.93 | 0.806 (0.629 | |
| | to 14.55) | to 15.87) | to 1.031) | |

Median follow-up time = 22.28 in months
^{a}Interaction test from the Cox proportional hazard model including the factor, treatment effect and the treatment by factor interaction

### 2. Progression Free Survival Based on Tumor Assessment by the IRC

The final analysis for PFS was performed at the time of the second interim analysis of OS (i.e. cut off date=6 May 2010). Improvement in progression free survival (PFS) was demonstrated in patients of the ziv-aflibercept treatment arm compared to patients in the placebo treatment arm (stratified HR: 0.758, 99.99% Cl: 0.578 to 0.995; p=0.00007). Median PFS was 6.90 months in the ziv-aflibercept arm and 4.67 months in the placebo arm (Table 11).

TABLE 11
PFS based on tumor assessment by the IRC (months) - Kaplan-Meier
survival estimates by treatment group - Stratified according to
stratification factors at randomization (IVRS) - ITT population

| | Placebo/Folfiri | Ziv-aflibercept/Folfiri |
|---|---|---|
| Time to Event or Censoring | (N = 614) | (N = 612) |
| Overall | | |
| Number of events, n/N(%) | 454/614 (73.9%) | 393/612 (64.2%) |
| Median PFS (99.99% CI) | 4.67 (4.074 | 6.90 (5.881 |
| (months) | to 5.552) | to 7.852) |
| Number at risk | | |
| 3 months | 355 | 420 |
| 6 months | 171 | 247 |
| 9 months | 94 | 99 |
| 12 months | 46 | 43 |
| 18 months | 9 | 7 |
| Probability of surviving | | |
| (99.99% CI) | | |
| 3 months | 0.664 (0.587 | 0.793 (0.727 |
| | to 0.741) | to 0.859) |
| 6 months | 0.390 (0.306 | 0.573 (0.488 |
| | to 0.475) | to 0.659) |
| 9 months | 0.254 (0.174 | 0.313 (0.222 |
| | to 0.334) | to 0.404) |
| 12 months | 0.146 (0.076 | 0.166 (0.085 |
| | to 0.216) | to 0.246) |
| 18 months | 0.043 (0.000 | 0.051 (0.000 |
| | to 0.091) | to 0.108) |
| Stratified Log-Rank | — | 0.00007 |
| test p-value^{a} | | |
| vs Placebo/Folfiri | | |
| Stratified Hazard | — | 0.758 (0.578 |
| ratio (99.99% CI)^{a} | | to 0.995) |
| vs Placebo/Folfiri | | |

Cutoff date = 6 MAY 2010
^{a}Stratified on ECOG Performance Status (0 vs 1 vs 2) and Prior Bevacizumab (yes vs no) according to IVRS
Significance threshold is set to 0.0001.

### Subgroup Analyses of Progression Free Survival

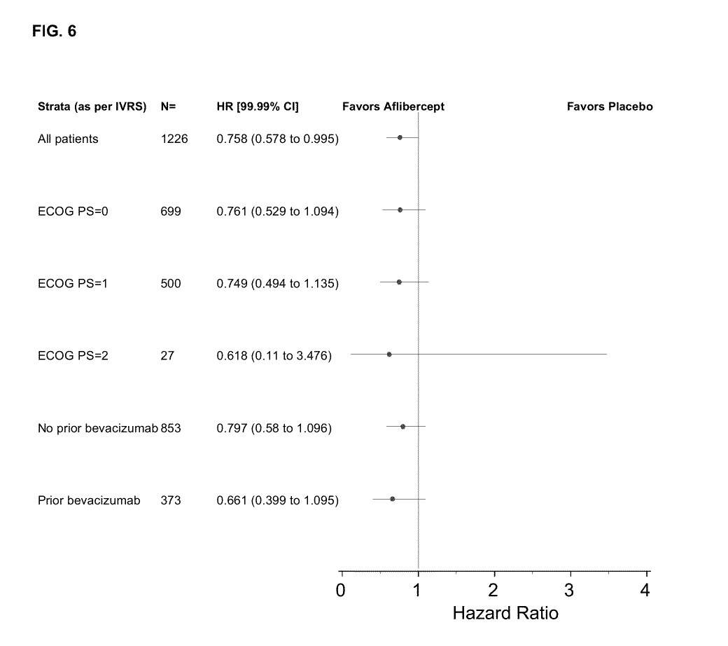

Progression free survival (PFS) was analyzed in subgroups as illustrated in Table 12 and in FIG. 6. No interaction between treatment arms and stratification factors was observed (Table 12).

TABLE 12
PFS based on tumor assessment by the IRC (months) - Summary of subgroup
analyses - By stratification factors as per IVRS - ITT population

| | Placebo/Folfiri | Ziv-aflibercept/Folfiri | Hazard Ratio | |
|---|---|---|---|---|
| | Median (Months) | Median (Months) | (99.99% CI) vs | P-value for |
| | (99.99% CI) | (99.99% CI) | Placebo/Folfiri | interaction^{a} |
| All patients | 4.7 (4.07 | 6.9 (5.88 | 0.758 (0.578 | |
| | to 5.55) | to 7.85) | to 0.995) | |
| Prior | | | | |
| bevacizumab | | | | |
| No | 5.4 (4.17 | 6.9 (5.82 | 0.797 (0.58 | 0.6954 |
| | to 6.70) | to 8.15) | to 1.096) | |
| Yes | 3.9 (2.86 | 6.7 (4.76 | 0.661 (0.399 | |
| | to 5.42) | to 8.74) | to 1.095) | |
| ECOG PS | | | | |
| 0 | 5.4 (4.24 | 7.2 (6.37 | 0.761 (0.529 | 0.1958 |
| | to 6.77) | to 8.87) | to 1.094) | |
| 1 | 4.1 (2.83 | 5.6 (4.60 | 0.749 (0.494 | |
| | to 5.55) | to 7.46) | to 1.135) | |
| 2 | 2.0 (1.18 | 2.7 (0.53 | 0.618 (0.11 | |
| | to 5.75) | to 12.88) | to 3.476) | |

Cutoff date = 6 MAY 2010
^{a}Interaction test from the Cox proportional hazard model including the factor, treatment effect and the treatment by factor interaction

For PFS, no significant interaction was shown between treatment arms and demographic variables or regions.

Treatment effect for PFS was consistent across subgroups with regards to baseline characteristics at study entry. Of note, the interaction between treatment arms and the presence of liver metastasis factor, that was noted on OS, was also significant at 10% level, indicating a higher treatment effect ‘in liver metastasis only’ group (HR (99.99% Cl): 0.547 (0.313 to 0.956)) than in ‘no liver metastasis, or other metastases’ group (HR (99.99% Cl): 0.839 (0.617 to 1.143)) (quantitative interaction, p=0.0076).

Results of the two sensitivity analyses for PFS were consistent with those of the primary PFS analysis. Moreover, adherence to the protocol-defined schedule for tumor assessment was assessed and showed no imbalance between treatment arms.

### 3. Overall Response Rate

Overall response rate—IRC reviewed—was significantly higher in the ziv-aflibercept treatment arm when compared to the placebo treatment arm: 19.8% (95% Cl: 16.4% to 23.2%) vs 11.1% (95% Cl: 8.5% to 13.8%) respectively (p=0.0001) (Table 13).

TABLE 13
Summary of overall objective response rate by IRC -
Evaluable patient population for response rate

| | Placebo/Folfiri | | Ziv-aflibercept/Folfiri | |
|---|---|---|---|---|
| | (N = 530) | | (N = 531) | |
| Best Overall | | | | |
| Response [n(%)] | | | | |
| Complete response | 2 | (0.4%) | 0 | |
| Partial response | 57 | (10.8%) | 105 | (19.8%) |
| Stable disease | 344 | (64.9%) | 350 | (65.9%) |
| Progressive disease | 114 | (21.5%) | 55 | (10.4%) |
| Not evaluable | 13 | (2.5%) | 21 | (4.0%) |
| Overall Response | | | | |
| Responders (Complete | 59 | (11.1%) | 105 | (19.8%) |
| response or | | | | |
| Partial response) | | | | |

| 95% CI^{a} | 8.5% to 13.8% | 16.4% to 23.2% |
|---|---|---|
| Stratified Cochran-Mantel- | — | 0.0001 |
| Haenszel test p-value^{b} | | |
| Vs Placebo/Folfiri | | |

^{a}estimated by Normal approximation
^{b}Stratified on ECOG Performance Status (0 vs 1 vs 2) and Prior Bevacizumab (yes vs no) according to IVRS.

### 4. Further Anti-Cancer Therapy

Overall 60% of patients in both treatment groups received further antitumor therapies (Table 14).

TABLE 14
Summary of first further anti-cancer therapies - ITT population

| | | | Ziv-aflibercept/ | |
|---|---|---|---|---|
| | Placebo/Folfiri | | Folfiri | |
| | (N = 614) | | (N = 612) | |
| At least one further | | | | |
| therapy [n(%)] | | | | |
| Yes | 366 | (59.6%) | 364 | (59.5%) |
| No | 248 | (40.4%) | 248 | (40.5%) |
| Type of first further | | | | |
| therapy [n(%)] | | | | |
| Systemic anti-cancer treatment | 303/366 | (82.8%) | 296/364 | (81.3%) |
| Radiotherapy | 43/366 | (11.7%) | 34/364 | (9.3%) |
| Surgery | 20/366 | (5.5%) | 34/364 | (9.3%) |
| Time from last IV to first | | | | |
| further systemic anti-cancer | | | | |
| therapy (months)^{a} | | | | |
| Number | 297 | | 293 | |
| Mean (SD) | 1.87 | (1.71) | 2.37 | (2.45) |
| Median | 1.35 | | 1.58 | |
| Min:Max | 0.3:14.0 | | 0.2:20.5 | |
| Time from last IV to first | | | | |
| further radiotherapy (months)^{a} | | | | |
| Number | 43 | | 33 | |
| Mean (SD) | 3.02 | (3.86) | 3.25 | (3.38) |
| Median | 1.31 | | 2.07 | |
| Min:Max | 0.4:16.5 | | 0.6:14.6 | |
| Time from last IV to first | | | | |
| further surgery (months)^{a} | | | | |
| Number | 20 | | 34 | |
| Mean (SD) | 1.62 | (1.41) | 2.42 | (2.08) |

| | | |
|---|---|---|
| Median | 1.15 | 1.48 |
| Min:Max | 0.4:7.2 | 0.2:8.5 |

Systemic anti-cancer therapies include chemotherapy and biologies. Only the earliest date of further therapy in each category (systemic anti-cancer treatment, radiotherapy or surgery) is kept
^{a}Time from last IV to first futher therapy is not calculated for patients randomized but not treated.

About 32% of patients in each group receive further anticancer treatment that includes a “biologic (Table 15).

TABLE 15
Summary of all further anti-cancer therapies - ITT population

| | | | Ziv-aflibercept/ | |
|---|---|---|---|---|
| | Placebo/Folfiri | | Folfiri | |
| | (N = 614) | | (N = 612) | |
| Any further therapy | 366 | (59.6%) | 364 | (59.5%) |
| Surgery | 31 | (5.0%) | 47 | (7.7%) |
| Radiotherapy | 81 | (13.2%) | 79 | (12.9%) |
| Systemic anti-cancer treatment | 329 | (53.6%) | 329 | (53.8%) |
| Biologies/Small molecules | 197 | (32.1%) | 195 | (31.9%) |
| Cetuximab | 91 | (14.8%) | 108 | (17.6%) |
| Bevacizumab | 75 | (12.2%) | 55 | (9.0%) |
| Panitumumab | 52 | (8.5%) | 52 | (8.5%) |
| Other | 14 | (2.3%) | 21 | (3.4%) |
| Chemotherapy | 297 | (48.4%) | 287 | (46.9%) |
| Fluoropyrimidine | 233 | (37.9%) | 223 | (36.4%) |
| Irinotecan | 160 | (26.1%) | 174 | (28.4%) |
| Other | 79 | (12.9%) | 71 | (11.6%) |
| Oxaliplatin | 66 | (10.7%) | 53 | (8.7%) |
| Other^{a} | 6 | (1.0%) | 5 | (0.8%) |

^{a}include patients randomized in placebo control trials for whom exact nature of the treatment is unknown
A patient can be counted both in chemotherapy and biologies (categories can not be added).

### 5. Safety

### Adverse Events

Treatment emergent adverse events, all grades, were reported in nearly 100% of the patients in both treatment arms, whereas occurrence of grade 3-4 events was greater in the ziv-aflibercept treatment arm (83.5% vs 62.5%).

The rate of permanent discontinuation of study treatment due to adverse events was higher in the ziv-aflibercept treatment arm (26.8% vs 12.1%). A similar pattern was observed for premature treatment discontinuation due to adverse events (19.5% vs 2.8%). Premature treatment discontinuation corresponds to an earlier discontinuation of either FOLFIRI, ziv-aflibercept/placebo being continued, or ziv-aflibercept/placebo, FOLFIRI being continued.

Within 30 days of last dosing, respectively 37 (6.1%) and 29 (4.8%) patients in the ziv-aflibercept and placebo arm, respectively, experienced, adverse events that eventually led to death within 30 days (28 vs 17 in the ziv-aflibercept and placebo arm, respectively) or after 30 days (9 vs 12 in the placebo and ziv-aflibercept arm, respectively) of last dosing. These included death due to disease progression.

A summary of safety data is illustrated in Table 16, Table 17 and Table 18.

TABLE 16
Summary of the most frequent TEAEs: incidence ≧20% in ziv-aflibercept arm
or (incidence <20% in ziv-aflibercept arm and Δ all grades ≧5%) - Safety population

| | | | Ziv- | | | | |
|---|---|---|---|---|---|---|---|
| % of | Placebo/ | | aflibercept/ | | | | |
| patients (in | Folfiri | | Folfiri | | | 5 ≦ Δ < 10 | |
| the safety | N = 605 | | N = 611 | | Δ ≧ 10% | % | Δ ≧ 2% |
| population) | All Gr | Gr 3/4 | All Gr | Gr 3/4 | all Gr | all Gr | Gr 3/4 |
| | Incidence ≧20% (ziv-aflibercept arm) | | | | | | |
| Diarrhea | 56.5 | 7.8 | 69.2 | 19.3 | X | | X |
| (PT) | | | | | | | |
| Asthenic | 50.2 | 10.6 | 60.4 | 16.9 | X | | X |
| condition | | | | | | | |
| (HLT) | | | | | | | |
| Stomatitis & | 34.9 | 5.0 | 54.8 | 13.7 | X | | X |
| ulceration | | | | | | | |
| (HLT) | | | | | | | |
| Nausea (PT) | 54.0 | 3.0 | 53.4 | 1.8 | | | |
| Infections | 32.7 | 6.9 | 46.2 | 12.3 | X | | X |
| (SOC) | | | | | | | |
| Hypertension | 10.7 | 1.5 | 41.4 | 19.3 | X | | X |
| (grouping) | | | | | | | |
| GI and | 29.1 | 3.3 | 34.0 | 5.4 | | | |
| abdominal | | | | | | | |
| pains (HLT) | | | | | | | |
| Vomiting | 33.4 | 3.5 | 32.9 | 2.8 | | | |
| (PT) | | | | | | | |
| Decrease | 23.8 | 1.8 | 31.9 | 3.4 | | X | |
| appetite | | | | | | | |
| (PT) | | | | | | | |
| Weight | 14.4 | 0.8 | 31.9 | 2.6 | X | | |
| decrease | | | | | | | |
| (PT) | | | | | | | |
| Epistaxis | 7.4 | 0 | 27.7 | 0.2 | X | | |
| (PT) | | | | | | | |
| Alopecia | 30.1 | NA | 26.8 | NA | | | |
| (PT) | | | | | | | |
| Dysphonia | 3.3 | 0 | 25.4 | 0.5 | X | | |
| (PT) | | | | | | | |
| Musculoskeletal | 21.2 | 2.3 | 23.1 | 1.3 | | | |
| & | | | | | | | |
| connective | | | | | | | |
| pain & | | | | | | | |
| discomfort | | | | | | | |
| (HLT) | | | | | | | |
| Constipation | 24.6 | 1.0 | 22.4 | 0.8 | | | |
| (PT) | | | | | | | |
| Headache | 8.8 | 0.3 | 22.3 | 1.6 | X | | |
| (PT) | | | | | | | |
| | Incidence <20% (ziv-aflibercept arm) and Δ all grades ≧5% | | | | | | |
| Palmar | 4.3 | 0.5 | 11.0 | 2.8 | | X | |
| plantar | | | | | | | |
| erythrodysaesthesia | | | | | | | |
| (PT) | | | | | | | |
| Dehydration | 3.0 | 1.3 | 9.0 | 4.3 | | X | |
| (PT) | | | | | | | |
| Skin | 2.8 | 0 | 8.2 | 0 | | X | |
| hyperpigmentation | | | | | | | |
| (PT) | | | | | | | |

Medra classification: SOC (system organ class), HLT (high level term), PT (Preferred term).
Grouping: grouping of selected PTs
Δ: difference in incidence in ziv-aflibercept arm compared to placebo

TABLE 17
Overview of safety, number (%) of patients - Safety population

| | | Ziv-aflibercept/ |
|---|---|---|
| | Placebo/Folfiri | Folfiri |
| | (N = 605) | (N = 611) |
| Patients with any TEAE | 592 (97.9%) | 606 (99.2%) |
| Patients with any grade 3-4 TEAE | 378 (62.5%) | 510 (83.5%) |
| Patients with any serious TEAE | 198 (32.7%) | 294 (48.1%) |
| Patients with any TEAE | 29 (4.8%) | 37 (6.1%) |
| leading to death | | |
| Patients with any related | 3 (0.5%) | 6 (1.0%) |
| TEAE leading to death | | |
| Patients with any TEAE | 73 (12.1%) | 164 (26.8%) |
| leading to permanent | | |
| treatment discontinuation | | |
| Patients with any TEAE | 17 (2.8%) | 119 (19.5%) |
| leading to premature | | |
| treatment discontinuation | | |

Note:
Adverse Events are reported using MedDRA version MEDDRA13.1 and graded using NCI CTC Version 3.0.

TABLE 18
Selected Adverse Reactions and Laboratory Findings:

| | Placebo/ | | ZALTRAP/ | |
|---|---|---|---|---|
| | FOLFIRI | | FOLFIRI | |
| | (N = 605) | | (N = 611) | |
| Primary System Organ | All | Grades | All | Grades |
| Class Preferred Term (%) | grades | 3-4 | grades | 3-4 |
| Infections and infestations | | | | |
| Urinary Tract Infection | 6% | 0.8% | 9% | 0.8% |
| Blood and lymphatic | | | | |
| system disorders | | | | |
| Leukopenia | 72% | 12% | 78% | 16% |
| Neutropenia | 57% | 30% | 67% | 37% |
| Thrombocytopenia | 35% | 2% | 48% | 3% |
| Metabolism and | | | | |
| nutrition disorders | | | | |
| Decreased Appetite | 24% | 2% | 32% | 3% |
| Dehydration | 3% | 1% | 9% | 4% |
| Nervous system disorders | | | | |
| Headache | 9% | 0.3% | 22% | 2% |
| Vascular disorders | | | | |
| Hypertension | 11% | 1.5% | 41% | 19% |
| Respiratory, thoracic and | | | | |
| mediastinal disorders | | | | |
| Epistaxis | 7% | 0 | 28% | 0.2% |
| Dysphonia | 3% | 0 | 25% | 0.5% |
| Dyspnea | 9% | 0.8% | 12% | 0.8% |
| Oropharyngeal Pain | 3% | 0 | 8% | 0.2% |
| Rhinorrhea | 2% | 0 | 6% | 0 |
| Gastrointestinal disorders | | | | |
| Diarrhea | 57% | 8% | 69% | 19% |
| Stomatitis | 33% | 5% | 50% | 13% |
| Abdominal Pain | 24% | 2% | 27% | 4% |
| Abdominal Pain Upper | 8% | 1% | 11% | 1% |
| Hemorrhoids | 2% | 0 | 6% | 0 |
| Rectal Hemorrhage | 2% | 0.5% | 5% | 0.7% |
| Proctalgia | 2% | 0.3% | 5% | 0.3% |
| Skin and subcutaneous | | | | |
| tissue disorders | | | | |
| Palmar-Plantar | 4% | 0.5% | 11% | 3% |
| Erythrodysesthesia Syndrome | | | | |
| Skin Hyperpigmentation | 3% | 0 | 8% | 0 |
| Renal and urinary disorders | | | | |
| Proteinuria* | 41% | 1% | 62% | 8% |
| Serum creatinine increased | 19% | 0.5% | 23% | 0 |
| General disorders and | | | | |
| administration site conditions | | | | |
| Fatigue | 39% | 8% | 48% | 13% |
| Asthenia | 13% | 3% | 18% | 5% |
| Investigations | | | | |
| AST increased | 54% | 2% | 62% | 3% |
| ALT increased | 39% | 2% | 50% | 3% |
| Weight decreased | 14% | 0.8% | 32% | 3% |

Note:
Adverse Reactions are reported using MedDRA version MEDDRA13.1 and graded using NCI CTC version 3.0
*Compilation of clinical and laboratory data

### 5. Conclusions

The study met its primary endpoint, with a significant improvement in overall survival in the ziv-aflibercept arm when compared to placebo.

In addition, a significant improvement was demonstrated on secondary efficacy endpoints (PFS and RR).

The safety profile was qualitatively consistent with that of anti-VEGF treatment with enhancement of known toxicities of the background chemotherapy (such as diarrhea, stomatitis, infections, neutropenia/neutropenic complications).

### Example 2

### US Approval: Full Prescribing Information

### Full Prescribing Information

### WARNING: HEMORRHAGE, GASTROINTESTINAL PERFORATION, COMPROMISED WOUND HEALING

Hemorrhage: Severe and sometimes fatal hemorrhage, including gastrointestinal (GI) hemorrhage, has been reported in the patients who have received ZALTRAP in combination with FOLFIRI. Monitor patients for signs and symptoms of GI bleeding and other severe bleeding. Do not administer ZALTRAP to patients with severe hemorrhage [see Dosage and Administration (2.4 Warnings and Precautions (5.1)].
Gastrointestinal Perforation: Gastrointestinal (GI) perforation including fatal GI perforation can occur in patients receiving ZALTRAP. Discontinue ZALTRAP therapy in patients who experience GI perforation [see Dosage and Administration (2.2), Warnings and Precautions (5.2)].
Compromised Wound Healing: Severe compromised wound healing can occur in patients receiving ZALTRAP/FOLFIRI. Discontinue ZALTRAP in patients with compromised wound healing. Suspend ZALTRAP for at least 4 weeks prior to elective surgery, and do not resume ZALTRAP for at least 4 weeks following major surgery and until the surgical wound is fully healed [see Dosage and Administration (2.2), Warnings and Precautions (5.3)].

### 1 Indications and Usage

ZALTRAP, in combination with 5-fluorouracil, leucovorin, irinotecan-(FOLFIRI), is indicated for patients with metastatic colorectal cancer (mCRC) that is resistant to or has progressed following an oxaliplatin-containing regimen [see Clinical Studies (14)].

### 2 Dosage and Administration

### 2.1 Recommended Dose and Schedule

Administer ZALTRAP 4 mg per kg as an intravenous (IV) infusion over 1 hour every two weeks. Administer ZALTRAP prior to any component of the FOLFIRI regimen on the day of treatment [see Clinical Studies (14)].

Continue ZALTRAP until disease progression or unacceptable toxicity.

### 2.2 Dose Modification/Treatment Delay Recommendations

### Discontinue ZALTRAP for:

Severe hemorrhage [see Boxed Warning, Warnings and Precautions (5.1)]
Gastrointestinal perforation [see Boxed Warning, Warnings and Precautions (5.2)]
Compromised wound healing [see Boxed Warning, Warnings and Precautions (5.3)]
Fistula formation [see Warnings and Precautions (5.4)]
Hypertensive crisis or hypertensive encephalopathy [see Warnings and Precautions (5.5)]
Arterial thromboembolic events [see Warnings and Precautions (5.6)]
Nephrotic syndrome or thrombotic microangiopathy (TMA) [see Warnings and Precautions (5.7)]
Reversible posterior leukoencephalopathy syndrome (RPLS) [see Warnings and Precautions (5.10)]

### Temporarily Suspend ZALTRAP:

At least 4 weeks prior to elective surgery [see Warnings and Precautions (5.3)]
For recurrent or severe hypertension, until controlled. Upon resumption, permanently reduce the ZALTRAP dose to 2 mg per kg [see Warnings and Precautions (5.5)].
For proteinuria of 2 grams per 24 hours. Resume when proteinuria is less than 2 grams per 24 hours. For recurrent proteinuria, suspend ZALTRAP until proteinuria is less than 2 grams per 24 hours and then permanently reduce the ZALTRAP dose to 2 mg per kg [see Warnings and Precautions (5.7)].

For toxicities related to irinotecan, 5-fluorouracil (5-FU), or leucovorin, refer to the current respective prescribing information.

### 2.3 Preparation for Administration

Inspect vials visually prior to use. ZALTRAP is a clear, colorless to pale yellow solution. Do not use vial if the solution is discolored or cloudy or if the solution contains particles. Do not re-enter the vial after the initial puncture. Discard any unused portion left in the vial. Withdraw the prescribed dose of ZALTRAP and dilute in 0.9% sodium chloride solution, USP or 5% dextrose solution for injection, USP to achieve a final concentration of 0.6-8 mg/mL. Use polyvinyl chloride (PVC) infusion bags containing bis (2-ethylhexyl) phthalate (DEHP) or polyolefin infusion bags.

Store diluted ZALTRAP at 2-8° C. (36-46° F.) for up to 4 hours. Discard any unused portion left in the infusion bag.

### 2.4 Administration

Administer the diluted ZALTRAP solution as an intravenous infusion over 1 hour through a 0.2 micron polyethersulfone filter. Do not use filters made of polyvinylidene fluoride (PVDF) or nylon.

Do not administer as an intravenous (IV) push or bolus.

Do not combine ZALTRAP with other drugs in the same infusion bag or intravenous line. Administer ZALTRAP using an infusion set made of one of the following materials:
PVC containing DEHP
DEHP free PVC containing trioctyl-trimellitate (TOTM)
polypropylene
polyethylene lined PVC
polyurethane

### 3 Dosage Forms and Strengths

### ZALTRAP is Available as:

100 mg per 4 mL (25 mg per mL) solution, single-use vial
200 mg per 8 mL (25 mg per mL) solution, single-use vial

### 4 Contraindications

### None

### 5 Warnings and Precautions

### 5.1 Hemorrhage

Patients treated with ZALTRAP have an increased risk of hemorrhage, including severe and sometimes fatal hemorrhagic events. In patients with mCRC, bleeding/hemorrhage (all grades) were reported in 38% of patients treated with ZALTRAP/FOLFIRI compared to 19% of patients treated with placebo/FOLFIRI. Grade 3-4 hemorrhagic events, including gastrointestinal hemorrhage, hematuria, and post-procedural hemorrhage, were reported in 3% of patients receiving ZALTRAP/FOLFIRI compared with 1% of patients receiving placebo/FOLFIRI. Severe intracranial hemorrhage and pulmonary hemorrhage/hemoptysis including fatal events have also occurred in patients receiving ZALTRAP.

Monitor patients for signs and symptoms of bleeding. Do not initiate ZALTRAP in patients with severe hemorrhage. Discontinue ZALTRAP in patients who develop severe hemorrhage [see Dosage and Administration (2.2)].

### 5.2 Gastrointestinal Perforation

Gastrointestinal (GI) perforation including fatal GI perforation can occur in patients receiving ZALTRAP. Across three Phase 3 placebo-controlled clinical studies (colorectal, pancreatic, and lung cancer populations), the incidence of GI perforation (all grades) was 0.8% for patients treated with ZALTRAP and 0.3% for patients treated with placebo. Grade 3-4 GI perforation events occurred in 0.8% of patients treated with ZALTRAP and 0.2% of patients treated with placebo.

Monitor patients for signs and symptoms of GI perforation. Discontinue ZALTRAP therapy in patients who experience GI perforation [see Dosage and Administration (2.2)].

### 5.3 Compromised Wound Healing

ZALTRAP impairs wound healing in animal models [see Nonclinical Toxicology (13.2)]. Grade 3 compromised wound healing was reported in 2 patients (0.3%) treated with ZALTRAP/FOLFIRI regimen and in none of the patients treated with placebo/FOLFIRI regimen.

Suspend ZALTRAP for at least 4 weeks prior to elective surgery. Do not resume ZALTRAP for at least 4 weeks following major surgery and until the surgical wound is fully healed. For minor surgery such as central venous access port placement, biopsy, and tooth extraction, ZALTRAP may be initiated/resumed once the surgical wound is fully healed. Discontinue ZALTRAP in patients with compromised wound healing [see Dosage and Administration (2.2)].

### 5.4 Fistula Formation

Fistula formation involving gastrointestinal and non-gastrointestinal sites occurs at a higher incidence in patients treated with ZALTRAP. In patients with mCRC, fistulas (anal, enterovesical, enterocutaneous, colovaginal, intestinal sites) were reported in 9 of 611 patients (1.5%) treated with ZALTRAP/FOLFIRI regimen and 3 of 605 patients (0.5%) treated with placebo/FOLFIRI regimen. Grade 3 GI fistula formation occurred in 2 patients treated with ZALTRAP (0.3%) and in 1 placebo-treated patient (0.2%).

Discontinue ZALTRAP therapy in patients who develop fistula [see Dosage and Administration (2.2)].

### 5.5 Hypertension

ZALTRAP increases the risk of Grade 3-4 hypertension. There is no clinical trial experience administering ZALTRAP to patients with NYHA class III or IV heart failure. In patients with mCRC, Grade 3 hypertension (defined as requiring adjustment in existing anti-hypertensive therapy or treatment with more than one drug) was reported in 1.5% of patients treated with placebo/FOLFIRI and 19% of patients treated with ZALTRAP/FOLFIRI. Grade 4 hypertension (hypertensive crisis) was reported in 1 patient (0.2%) treated with ZALTRAP/FOLFIRI. Among those patients treated with ZALTRAP/FOLFIRI developing Grade 3-4 hypertension, 54% had onset during the first two cycles of treatment.

Monitor blood pressure every two weeks or more frequently as clinically indicated during treatment with ZALTRAP. Treat with appropriate anti-hypertensive therapy and continue monitoring blood pressure regularly. Temporarily suspend ZALTRAP in patients with uncontrolled hypertension until controlled, and permanently reduce ZALTRAP dose to 2 mg per kg for subsequent cycles. Discontinue ZALTRAP in patients with hypertensive crisis or hypertensive encephalopathy [see Dosage and Administration (2.2)].

### 5.6 Arterial Thromboembolic Events

Arterial thromboembolic events (ATE), including transient ischemic attack, cerebrovascular accident, and angina pectoris, occurred more frequently in patients who have received ZALTRAP. In patients with mCRC, ATE was reported in 2.6% of patients treated with ZALTRAP/FOLFIRI and 1.7% of patients treated with placebo/FOLFIRI. Grade 3-4 events occurred in 11 patients (1.8%) treated with ZALTRAP/FOLFIRI and 4 patients (0.7%) treated with placebo/FOLFIRI.

Discontinue ZALTRAP in patients who experience an ATE [see Dosage and Administration (2.2)].

### 5.7 Proteinuria

Severe proteinuria, nephrotic syndrome, and thrombotic microangiopathy (TMA) occurred more frequently in patients treated with ZALTRAP. In patients with mCRC, proteinuria was reported in 62% patients treated with ZALTRAP/FOLFIRI compared to 41% patients treated with placebo/FOLFIRI. Grade 3-4 proteinuria occurred in 8% of patients treated with ZALTRAP/FOLFIRI to 1% of patients treated with placebo/FOLFIRI [see Adverse Reactions (6.1)]. Nephrotic syndrome occurred in 2 patients (0.5%) treated with ZALTRAP/FOLFIRI compared to none of the patients treated with placebo/FOLFIRI. TMA was reported in 3 of 2258 patients with cancer enrolled across completed studies.

Monitor proteinuria by urine dipstick analysis and urinary protein creatinine ratio (UPCR) for the development or worsening of proteinuria during ZALTRAP therapy. Obtain a 24-hour urine collection in patients with a UPCR greater than 1.

Suspend ZALTRAP administration for proteinuria 2 grams per 24 hours or more, and resume when proteinuria is less than 2 grams per 24 hours. If recurrent, suspend until proteinuria is less than 2 grams per 24 hours and then permanently reduce the ZALTRAP dose to 2 mg per kg. Discontinue ZALTRAP in patients who develop nephrotic syndrome or TMA [see Dosage and Administration (2.2)].

### 5.8 Neutropenia and Neutropenic Complications

A higher incidence of neutropenic complications (febrile neutropenia and neutropenic infection) occurred in patients receiving ZALTRAP. In patients with mCRC, Grade 3-4 neutropenia occurred in 37% of patients treated with ZALTRAP/FOLFIRI compared to 30% patients treated with placebo/FOLFIRI [see Adverse Reactions (6.1)]. Grade 3-4 febrile neutropenia occurred in 4% of patients treated with ZALTRAP/FOLFIRI compared to 2% of patients treated with placebo/FOLFIRI. Grade 3-4 neutropenic infection/sepsis occurred in 1.5% of patients treated with ZALTRAP/FOLFIRI and 1.2% of patients treated with placebo/FOLFIRI. Monitor CBC with differential count at baseline and prior to initiation of each cycle of ZALTRAP. Delay ZALTRAP/FOLFIRI until neutrophil count is at or above 1.5×109/L.

### 5.9 Diarrhea and Dehydration

The incidence of severe diarrhea is increased in patients treated with ZALTRAP/FOLFIRI. In patients with mCRC, Grade 3-4 diarrhea was reported in 19% of patients treated with ZALTRAP/FOLFIRI compared to 8% of patients treated with placebo/FOLFIRI. Grade 3-4 dehydration was reported in 4% of patients treated with ZALTRAP/FOLFIRI compared to 1% of patients treated with placebo/FOLFIRI [see Adverse Reactions (6.1)]. The incidence of diarrhea is increased in patients who are age 65 years or older as compared to patients younger than 65 years of age [see Geriatric Use (8.5)]. Monitor elderly patients closely for diarrhea.

### 5.10 Reversible Posterior Leukoencephalopathy Syndrome (RPLS)

RPLS (also known as posterior reversible encephalopathy syndrome) was reported in 0.5% of 3795 patients treated with ZALTRAP monotherapy or in combination with chemotherapy.

Confirm the diagnosis of RPLS with MRI and discontinue ZALTRAP in patients who develop RPLS. Symptoms usually resolve or improve within days, although some patients have experienced ongoing neurologic sequelae or death [see Dosage and Administration (2.2)].

### 6 Adverse Reactions

The following serious adverse reactions are discussed elsewhere in the labeling:
Hemorrhage [see Boxed Warning, Warnings and Precautions (5.1)]
Gastrointestinal Perforation [see Boxed Warning, Warnings and Precautions (5.2)]
Compromised Wound Healing [see Boxed Warning, Warnings and Precautions (5.3)]
Fistula Formation [see Warnings and Precautions (5.4)]
Hypertension [see Warnings and Precautions (5.5)]
Arterial Thromboembolic Events [see Warnings and Precautions (5.6)]
Proteinuria [see Warnings and Precautions (5.7)]
Neutropenia and Neutropenic Complications [see Warnings and Precautions (5.8)]
Diarrhea and Dehydration [see Warnings and Precautions (5.9)]
Reversible Posterior Leukoencephalopathy Syndrome (RPLS) [see Warnings and Precautions (5.10)]

### 6.1 Clinical Trial Experience

Because clinical trials are conducted under varying designs and in different patient populations, the adverse reaction rates reported in one clinical trial may not be easily compared to those rates reported in another clinical trial, and may not reflect the rates actually observed in clinical practice.

The safety of ZALTRAP in combination with FOLFIRI was evaluated in 1216 previously treated patients with metastatic colorectal cancer (Study 1) who were treated with ZALTRAP 4 mg per kg intravenous (N=611) or placebo (N=605) every two weeks (one cycle) in a randomized (1:1), double-blind, placebo-controlled Phase 3 study. Patients received a median of 9 cycles of ZALTRAP/FOLFIRI or 8 cycles of placebo/FOLFIRI.

The most common adverse reactions (all grades, ≧20% incidence) reported at a higher incidence (2% or greater between-arm difference) in the ZALTRAP/FOLFIRI arm, in order of decreasing frequency, were leukopenia, diarrhea, neutropenia, proteinuria, AST increased, stomatitis, fatigue, thrombocytopenia, ALT increased, hypertension, weight decreased, decreased appetite, epistaxis, abdominal pain, dysphonia, serum creatinine increased, and headache (see Table 1).

The most common Grade 3-4 adverse reactions (≧5%) reported at a higher incidence (2% or greater between-arm difference) in the ZALTRAP/FOLFIRI arm, in order of decreasing frequency, were neutropenia, diarrhea, hypertension, leukopenia, stomatitis, fatigue, proteinuria, and asthenia (see Table 1).

The most frequent adverse reactions leading to permanent discontinuation in 1% of patients treated with ZALTRAP/FOLFIRI regimen were asthenia/fatigue, infections, diarrhea, dehydration, hypertension, stomatitis, venous thromboembolic events, neutropenia, and proteinuria.

The ZALTRAP dose was reduced and/or omitted in 17% of patients compared to placebo-dose modification in 5% of patients. Cycle delays>7 days occurred in 60% of patients treated with ZALTRAP/FOLFIRI compared with 43% of patients treated with placebo/FOLFIRI.

The most common adverse reactions and laboratory abnormalities during study treatment in Study 1 where the incidence was ≧5% (all grades) in patients receiving ZALTRAP in combination with FOLFIRI and which occurred at ≧2% higher frequency in patients treated with ZALTRAP/FOLFIRI compared to placebo/FOLFIRI are shown in Table 19.

TABLE 19
Selected Adverse Reactions and Laboratory Findings in Study 1:

| | Placebo/ | | ZALTRAP/ | |
|---|---|---|---|---|
| | FOLFIRI | | FOLFIRI | |
| | (N = 605) | | (N = 611) | |
| Primary System Organ Class | All | Grades | All | Grades |
| Preferred Term (%) | grades | 3-4 | grades | 3-4 |
| Infections and infestations | | | | |
| Urinary Tract Infection | 6% | 0.80% | 9% | 0.80% |
| Blood and lymphatic | | | | |
| system disorders | | | | |
| Leukopenia | 72% | 12% | 78% | 16% |
| Neutropenia | 57% | 30% | 67% | 37% |
| Thrombocytopenia | 35% | 2% | 48% | 3% |
| Metabolism and | | | | |
| nutrition disorders | | | | |
| Decreased Appetite | 24% | 2% | 32% | 3% |
| Dehydration | 3% | 1% | 9% | 4% |
| Nervous system disorders | | | | |
| Headache | 9% | 0.30% | 22% | 2% |
| Vascular disorders | | | | |
| Hypertension | 11% | 1.50% | 41% | 19% |
| Respiratory, thoracic and | | | | |
| mediastinal disorders | | | | |
| Epistaxis | 7% | 0 | 28% | 0.20% |
| Dysphonia | 3% | 0 | 25% | 0.50% |
| Dyspnea | 9% | 0.80% | 12% | 0.80% |
| Oropharyngeal Pain | 3% | 0 | 8% | 0.20% |
| Rhinorrhea | 2% | 0 | 6% | 0 |
| Gastrointestinal disorders | | | | |
| Diarrhea | 57% | 8% | 69% | 19% |
| Stomatitis | 33% | 5% | 50% | 13% |
| Abdominal Pain | 24% | 2% | 27% | 4% |
| Abdominal Pain Upper | 8% | 1% | 11% | 1% |
| Hemorrhoids | 2% | 0 | 6% | 0 |
| Rectal Hemorrhage | 2% | 0.50% | 5% | 0.70% |
| Proctalgia | 2% | 0.30% | 5% | 0.30% |
| Skin and subcutaneous | | | | |
| tissue disorders | | | | |
| Palmar-Plantar | 4% | 0.50% | 11% | 3% |
| Erythrodysesthesia Syndrome | | | | |
| Skin Hyperpigmentation | 3% | 0 | 8% | 0 |
| Renal and urinary disorders | | | | |
| Proteinuria* | 41% | 1% | 62% | 8% |
| Serum creatinine increased | 19% | 0.50% | 23% | 0 |
| General disorders and | | | | |
| administration site conditions | | | | |
| Fatigue | 39% | 8% | 48% | 13% |
| Asthenia | 13% | 3% | 18% | 5% |
| Investigations | | | | |
| AST increased | 54% | 2% | 62% | 3% |
| ALT increased | 39% | 2% | 50% | 3% |
| Weight decreased | 14% | 0.8% | 32% | 3% |

Note:
Adverse Reactions are reported using MedDRA version MEDDRA13.1 and graded using NCI CTC version 3.0
*Compilation of clinical and laboratory data

Infections occurred at a higher frequency in patients receiving ZALTRAP/FOLFIRI (46%, all grades; 12%, Grade 3-4) than in patients receiving placebo/FOLFIRI (33%, all grades; 7%, Grade 3-4), including urinary tract infection, nasopharyngitis, upper respiratory tract infection, pneumonia, catheter site infection, and tooth infection.

In patients with mCRC, severe hypersensitivity reactions have been reported with ZALTRAP/FOLFIRI (0.3%) and placebo/FOLFIRI (0.5%).

In patients with mCRC, venous thromboembolic events (VTE), consisting primarily of deep venous thrombosis and pulmonary embolism, occurred in 9% of patients treated with ZALTRAP/FOLFIRI and 7% of patients treated with placebo/FOLFIRI. Grade 3-4 VTE occurred in 8% of patients treated with ZALTRAP/FOLFIRI and in 6% of patients treated with placebo/FOLFIRI. Pulmonary embolism occurred in 5% of patients treated with ZALTRAP/FOLFIRI and 3.4% of patients treated with placebo/FOLFIRI.

### 6.2 Immunogenicity

As with all therapeutic proteins, there is a potential for immunogenicity. In patients with various cancers across 15 studies, 1.4% (41/2862) of patients tested positive for anti-product antibody (APA) at baseline. The incidence of APA development was 3.1% (53/1687) in patients receiving intravenous ziv-aflibercept and 1.7% (19/1134) in patients receiving placebo. Among patients who tested positive for APA and had sufficient samples for further testing, neutralizing antibodies were detected in 17 of 48 ziv-aflibercept-treated patients and in 2 of 40 patients receiving placebo.

The mean free ziv-aflibercept trough concentrations were lower in patients with positive neutralizing antibodies than in the overall population. The impact of neutralizing antibodies on efficacy and safety could not be assessed based on limited available data.

Immunogenicity data are highly dependent on the sensitivity and specificity of the assay. Additionally, the observed incidence of antibody positivity in an assay may be influenced by several factors, including sample handling, timing of sample collection, concomitant medications, and underlying disease. For these reasons, comparison of the incidence of antibodies to ZALTRAP with the incidence of antibodies to other products may be misleading.

### 7 Drug Interactions

No dedicated drug-drug interaction studies have been conducted for ZALTRAP. No clinically important pharmacokinetic drug-drug interactions were found between ziv-aflibercept and irinotecan/SN-38 or 5-FU, based on cross-study comparisons and population pharmacokinetic analyses.

### 8 Use in Specific Populations

### 8.1 Pregnancy

### Pregnancy Category C

### Risk Summary

There are no adequate and well-controlled studies with ZALTRAP in pregnant women. ZALTRAP was embryotoxic and teratogenic in rabbits at exposure levels lower than human exposures at the recommended dose, with increased incidences of external, visceral, and skeletal

fetal malformations. ZALTRAP should be used during pregnancy only if the potential benefit justifies the potential risk to the fetus.

### Animal Data

Ziv-aflibercept produced embryo-fetal toxicity when administered every 3 days during organogenesis in pregnant rabbits at all intravenous doses tested, 3 mg per kg. Adverse embryo-fetal effects included increased incidences of postimplantation losses and external (including anasarca, umbilical hernia, diaphragmatic hernia and gastroschisis, cleft palate, ectrodactyly, and atresia), visceral (in the heart, great vessels, and arteries), and skeletal fetal malformations (including fused vertebrae, sternebrae, and ribs; supernumerary arches and ribs, and incomplete ossification). Administration of the 3 mg per kg dose to rabbits resulted in systemic exposure (AUC) that was approximately 30% of the AUC in patients at the recommended dose. The incidence and severity of fetal anomalies increased with increasing dose.

### 8.3 Nursing Mothers

It is not known whether ZALTRAP is excreted in human milk. Because many drugs are excreted in human milk and because of the potential for serious adverse reactions in nursing infants from ZALTRAP, a decision should be made whether to discontinue nursing or discontinue the drug, taking into account the importance of the drug to the mother.

### 8.4 Pediatric Use

The safety and effectiveness in pediatric patients have not been established.

### 8.5 Geriatric Use

Of the 611 patients with mCRC, patients treated with ZALTRAP/FOLFIRI, 205 (34%) were 65 years or older, and 33 (5%) were 75 years or older. Elderly patients (≧65 years of age) experienced higher incidences (≧5%) of diarrhea, dizziness, asthenia, weight decrease, and dehydration when compared to younger patients. Monitor elderly patients more closely for diarrhea and dehydration [see Warnings and Precautions (5.9)].

The effect of ZALTRAP on overall survival was similar in patients<65 years old and 65 years old who received ZALTRAP/FOLFIRI.

No dose adjustment of ZALTRAP is recommended for patients greater than or equal to 65 years of age.

### 8.6 Hepatic Impairment

No dedicated clinical studies have been conducted to evaluate the effect of hepatic impairment on the pharmacokinetics of ziv-aflibercept.

Based on a population PK analysis with data from 1507 patients, ziv-aflibercept exposure in patients with mild and moderate hepatic impairment were similar to those in patients with normal hepatic function [see Clinical Pharmacology (12.3)]. There are no data available for patients with severe hepatic impairment.

### 8.7 Renal Impairment

No dedicated clinical studies have been conducted to evaluate the effect of renal impairment on the pharmacokinetics of ziv-aflibercept.

Based on a population PK analysis with data from 1507 patients, ziv-aflibercept exposure in patients with mild, moderate, and severe renal impairment were similar to those in patients with normal renal function [see Clinical Pharmacology (12.3)].

### 8.8 Females and Males of Reproductive Potential

Male and female reproductive function and fertility may be compromised during treatment with ZALTRAP, as suggested by findings in monkeys [see Nonclinical Toxicology (13.1)]. These animal findings were reversible within 18 weeks after cessation of treatment. Females and males

of reproductive potential should use highly effective contraception during and up to a minimum of 3 months after the last dose of treatment.

### 10 Overdosage

There have been no cases of overdose reported with ZALTRAP. There is no information on the safety of ZALTRAP given at doses exceeding 7 mg per kg every 2 weeks or 9 mg per kg every 3 weeks.

### 11 Description

Ziv-aflibercept is a recombinant fusion protein consisting of Vascular Endothelial Growth Factor (VEGF)-binding portions from the extracellular domains of human VEGF Receptors 1 and 2 fused to the Fc portion of the human IgG1. Ziv-aflibercept is produced by recombinant DNA technology in a Chinese hamster ovary (CHO) K-1 mammalian expression system.

Ziv-aflibercept is a dimeric glycoprotein with a protein molecular weight of 97 kilodaltons (kDa) and contains glycosylation, constituting an additional 15% of the total molecular mass, resulting in a total molecular weight of 115 kDa.

ZALTRAP is a sterile, clear, colorless to pale yellow, non-pyrogenic, preservative-free, solution for administration by intravenous infusion. ZALTRAP is supplied in single-use vials of 100 mg per 4 ml and 200 mg per 8 ml formulated as 25 mg/mL ziv-aflibercept in polysorbate 20 (0.1%), sodium chloride (100 mM), sodium citrate (5 mM), sodium phosphate (5 mM), and sucrose (20%), in Water for Injection USP, at a pH of 6.2.

### 12 Clinical Pharmacology

### 12.1 Mechanism of Action

Ziv-aflibercept acts as a soluble receptor that binds to human VEGF-A (equilibrium dissociation constant KD of 0.5 pM for VEGF-A165 and 0.36 pM for VEGF-A121), to human VEGF-B (KD of 1.92 pM), and to human PIGF (KD of 39 pM for PIGF-2). By binding to these endogenous ligands, ziv-aflibercept can inhibit the binding and activation of their cognate receptors. This inhibition can result in decreased neovascularization and decreased vascular permeability. In animals, ziv-aflibercept was shown to inhibit the proliferation of endothelial cells, thereby inhibiting the growth of new blood vessels. Ziv-aflibercept inhibited the growth of xenotransplanted colon tumors in mice.

### 12.3 Pharmacokinetics

Plasma concentrations of free and VEGF-bound ziv-aflibercept were measured using specific enzyme-linked immunosorbent assays (ELISAs). Free ziv-aflibercept concentrations appear to exhibit linear pharmacokinetics in the dose range of 2-9 mg/kg. Following 4 mg/kg every two weeks intravenous administration of ZALTRAP, the elimination half-life of free ziv-aflibercept was approximately 6 days (range 4-7 days). Steady state concentrations of free ziv-aflibercept were reached by the second dose. The accumulation ratio for free ziv-aflibercept was approximately 1.2 after administration of 4 mg/kg every two weeks.

### Specific Populations

Based on a population pharmacokinetic analysis, age, race, and gender did not have a clinically important effect on the exposure of free ziv-aflibercept. Patients weighing≧100 kg had a 29% increase in systemic exposure compared to patients weighing 50 to 100 kg.

### Hepatic Impairment

Based on a population pharmacokinetic analysis which included patients with mild (total bilirubin>1.0×-1.5×ULN and any SGOT/AST, n=63) and moderate (total bilirubin>1.5×-3×ULN and any SGOT/AST, n=5) hepatic impairment, there was no effect of total bilirubin, aspartate amino transferase, and alanine amino transferase on the clearance of free ziv-aflibercept. There is no data available for patients with severe hepatic impairment (total bilirubin>3×ULN and any SGOT/AST).

### Renal Impairment

Based on a population pharmacokinetic analysis which included patients with mild (CLCR 50-80 mL/min, n=549), moderate (CLCR 30-50 mL/min, n=96), and severe renal impairment (CLCR<30

mL/min, n=5), there was no clinically important effect of creatinine clearance on the clearance of free ziv-aflibercept.

### 12.6 Cardiac Electrophysiology

The effect of 6 mg/kg intravenous ZALTRAP every three weeks on QTc interval was evaluated in 87 patients with solid tumors in a randomized, placebo-controlled study. No large changes in the mean QT interval from baseline (i.e., greater than 20 ms as corrected for placebo) based on Fridericia correction method were detected in the study. However, a small increase in the mean QTc interval (i.e., less than 10 ms) cannot be excluded due to limitations of the study design.

### 13 Nonclinical Toxicology

### 13.1 Carcinogenesis, Mutagenesis, Impairment of Fertility

No studies have been conducted to evaluate carcinogenicity or mutagenicity of ziv-aflibercept. Ziv-aflibercept impaired reproductive function and fertility in monkeys. In a 6-month repeatdose toxicology study in sexually mature monkeys, ziv-aflibercept inhibited ovarian function and follicular development, as evidenced by: decreased ovary weight, decreased amount of luteal tissue, decreased number of maturing follicles, atrophy of uterine endometrium and myometrium, vaginal atrophy, abrogation of progesterone peaks and menstrual bleeding. Alterations in sperm morphology and decreased sperm motility were noted in male monkeys. These effects were observed at all doses tested including the lowest dose tested, 3 mg per kg. Reversibility was observed within 18 weeks after cessation of treatment. Systemic exposure (AUC) with a 3 mg per kg per dose in monkeys was approximately 60% of the AUC in patients at the recommended dose.

13.2 Animal Toxicology and/or Pharmacology

Weekly/every two weeks intravenous administration of ziv-aflibercept to growing young adult (sexually mature) cynomolgus monkeys for up to 6 months resulted in changes in the bone (effects on growth plate and the axial and appendicular skeleton), nasal cavity (atrophy/loss of the septum and/or turbinates), kidney (glomerulopathy with inflammation), ovary (decreased number of maturing follicles, granulosa cells, and/or theca cells), and adrenal gland (decreased vacuolation with inflammation). Most ziv-aflibercept-related findings were noted from the lowest dose tested (3 mg per kg per dose) correlating to 60% of the AUC at the human recommended dose.

In another study in sexually immature cynomolgus monkeys (treated intravenous for 3 months), similar effects were observed. The skeletal and nasal cavity effects were not reversible after a post-dosing recovery period.

Repeated administration of ziv-aflibercept resulted in a delay in wound healing in rabbits. In full-thickness excisional and incisional skin wound models, ziv-aflibercept administration reduced fibrous response, neovascularization, epidermal hyperplasia/re-epithelialization, and tensile strength.

### 14 Clinical Studies

Study 1 was a randomized, double-blind, placebo-controlled study in patients with metastatic colorectal cancer (mCRC) who are resistant to or have progressed during or within 6 months of receiving oxaliplatin-based combination chemotherapy, with or without prior bevacizumab. A total of 1226 patients were randomized (1:1) to receive either ZALTRAP (N=612; 4 mg per kg as a 1 hour intravenous infusion on day 1) or placebo (N=614), in combination with 5-fluorouracil plus irinotecan [FOLFIRI: irinotecan 180 mg per m2 IV infusion over 90 minutes and leucovorin (dl racemic) 400 mg per m² intravenous infusion over 2 hours at the same time on day 1 using a Y-line, followed by 5-FU 400 mg per m² intravenous bolus, followed by 5-FU 2400 mg per m² continuous intravenous infusion over 46-hours]. The treatment cycles on both arms were repeated every 2 weeks. Patients were treated until disease progression or unacceptable toxicity. The primary efficacy endpoint was overall survival. Treatment assignment was stratified by the ECOG performance status (0 versus 1 versus 2) and according to prior therapy with bevacizumab (yes or no).

Demographics characteristics were similar between treatment arms. Of the 1226 patients randomized, the median age was 61 years, 59% were men, 87% were White, 7% were Asian, 3.5% were Black, and 98% had a baseline ECOG performance status (PS) of 0 or 1. Among the 1226 randomized patients, 89% and 90% of patients treated with placebo/FOLFIRI and ZALTRAP/FOLFIRI, respectively, received prior oxaliplatin-based combination chemotherapy in the metastatic/advanced setting. A total of 346 patients (28%) received bevacizumab in combination with the prior oxaliplatin-based treatment.

Overall efficacy results for the ZALTRAP/FOLFIRI regimen versus the placebo/FOLFIRI regimen are summarized in Table 20.

TABLE 20
Main efficacy outcome measures ^{a}

| | Placebo/ | ZALTRAP/ |
|---|---|---|
| | FOLFIRI | FOLFIRI |
| | (N = 614) | (N = 612) |
| Overall Survival | | |
| Number of deaths, n (%) | 460 (74.9%) | 403 (65.8%) |
| Median overall survival (95% CI) (months) | 12.06 (11.07 | 13.50 (12.52 |
| | to 13.08) | to 14.95) |
| Stratified Hazard ratio (95% CI) | 0.817 (0.714 to 0.935) | |
| Stratified Log-Rank test p-value | 0.0032 | |
| Progression Free Survival (PFS)a | | |
| Number of events, n (%) | 454 (73.9%) | 393 (64.2%) |
| Median PFS (95% CI) (months) | 4.67 (4.21 | 6.90 (6.51 |
| | to 5.36) | to 7.20) |
| Stratified Hazard ratio (95% CI) | 0.758 (0.661 to 0.869) | |
| Stratified Log-Rank test p-value b | 0.00007 | |
| Overall Response Rate (CR + PR) (95% | 11.1 (8.5 | 19.8 (16.4 |
| CI) (%)c | to 13.8) | to 23.2) |

| | |
|---|---|
| Stratified Cochran-Mantel-Haenszel test | 0.0001 |
| p-value | |

aPFS (based on tumor assessment by the IRC): Significance threshold is set to 0.0001.
b Stratified on ECOG Performance Status (0 vs 1 vs 2) and Prior Bevacizumab (yes vs no)
cOverall objective response rate by IRC

Planned subgroup analyses for overall survival based on stratification factors at randomization yielded an HR of 0.86 (95% Cl: 0.68 to 1.1) in patients who received prior bevacizumab and an HR of 0.79 (95% Cl: 0.67 to 0.93) in patients without prior bevacizumab exposure.

### 16 how Supplied/Storage and Handling

### 16.1 How Supplied

ZALTRAP is supplied in 5 mL and 10 mL vials containing ziv-aflibercept at a concentration of 25 mg/mL.

NDC 0024-5840-01: carton containing one (1) single-use vial of 100 mg per 4 mL (25 mg/mL)

NDC 0024-5840-03: carton containing three (3) single-use vials of 100 mg per 4 mL (25 mg/mL)

NDC 0024-5841-01: carton containing one (1) single-use vial of 200 mg per 8 mL (25 mg/mL)

### 16.2 Storage and Handling

Store ZALTRAP vials in a refrigerator at 2 to 8° C. (36 to 46° F.). Keep the vials in the original outer carton to protect from light.

### 17 Patient Counseling Information

### Advise Patients:

That ZALTRAP can cause severe bleeding. Advise patients to contact their health care provider for bleeding or symptoms of bleeding including lightheadedness.
That ZALTRAP increases the risk of compromised wound healing. Instruct patients not to undergo surgery or procedures (including tooth extractions) without discussing first with their health care provider.
That ZALTRAP can cause or exacerbate existing hypertension. Advise patients to undergo routine blood pressure monitoring and to contact their health care provider if blood pressure is elevated or if symptoms from hypertension occur including severe headache, lightheadedness, or neurologic symptoms.
To notify the health care provider of severe diarrhea, vomiting, or severe abdominal pain.
To notify their health care provider of fever or other signs of infection.
Of an increased risk of arterial thromboembolic events.
Of the potential risks to the fetus or neonate using ZALTRAP during pregnancy or nursing and of the need to use highly effective contraception in both males and females during and for at least 3 months following last dose of ZALTRAP therapy. Advise the patient to immediately contact the healthcare provider if they or their partner becomes pregnant during treatment with ZALTRAP.

1. An article of manufacture comprising:
a) a packaging material
b) a polypeptide of SEQ ID NO:1, or a biosimilar thereof, and
c) a label or package insert contained within said packaging material indicating that:
the polypeptide or biosimilar thereof should not be administered to patients with severe haemorrhage, and/or
the therapy should be discontinued in patients who experience gastrointestinal perforation, and/or
the therapy should be discontinued in patients with compromised wound healing.

2-17. (canceled)

18. A method of promoting the use of a polypeptide of SEQ ID NO:1, or a biosimilar thereof, the method comprising the step of conveying to a recipient at least one message selected from the group consisting of:
a) the polypeptide, or biosimilar thereof, should not be administered to patients with severe haemorrhage, and/or
b) the polypeptide, or biosimilar thereof, should be discontinued in patients who experience gastrointestinal perforation, and/or
c) the polypeptide, or biosimilar thereof, should be discontinued in patients with compromised wound healing.

19. A method of treating cancer or cancer symptom in a patient in need thereof, said method comprising assessing whether said patient presents severe haemorrhage, and, if not, administering to said patient a therapeutically effective amount of a polypeptide of SEQ ID NO:1, or biosimilar thereof.

20. A method of treating cancer or cancer symptom in a patient in need thereof, said method comprising administering to said patient a therapeutically effective amount of a polypeptide of SEQ ID NO:1, or biosimilar thereof, wherein:
a) the polypeptide, or biosimilar thereof, is not administered to patients with severe haemorrhage, and/or
b) the administration of the polypeptide, or biosimilar thereof, is discontinued in patients who experience gastrointestinal perforation, and/or
c) the administration of the polypeptide, or biosimilar thereof, is discontinued in patients with compromised wound healing.

21. A method of treating Colorectal Cancer (CRC) or Colorectal Cancer (CRC) symptom in a patient in need thereof, said method comprising administering to said patient therapeutically effective amounts of a polypeptide of SEQ ID NO:1, or biosimilar thereof, leucovorin, 5-fluorouracil (5-FU) and irinotecan wherein:
a) the polypeptide, or biosimilar thereof, is not administered to patients with severe haemorrhage, and/or
b) the administration of the polypeptide, or biosimilar thereof, is discontinued in patients who experience gastrointestinal perforation, and/or
c) the administration of the polypeptide, or biosimilar thereof, is discontinued in patients with compromised wound healing.

22. A method according to claim 21 wherein said patient has already been treated for the CRC or CRC symptom.

23. A method according to claim 22 wherein said patient has previously been treated with chemotherapy, radiotherapy or surgery.

24. A method according to claim 21 wherein said patient has previously been treated with therapy based on oxaliplatin or bevacizumab.

25. A method according to claim 24 wherein said prior therapy has failed.

26. A method according to claim 21 wherein CRC is a Metastatic CRC.

27. A method according to claim 21 wherein said method is indicated for patients with metastatic colorectal cancer (mCRC) that is resistant to or has progressed following an oxaliplatin-containing regimen.

28. A method according to claim 21 wherein leucovorin at a dosage comprised between about 200 mg/m² and about 600 mg/m², 5-fluorouracil (5-FU) at a dosage comprised between about 2000 mg/m² and about 4000 mg/m², irinotecan at a dosage comprised between about 100 mg/m² and about 300 mg/m² and the polypeptide of SEQ ID NO:1, or biosimilar thereof, at a dosage comprised between about 1 mg/kg and about 10 mg/kg are administered to patient.

29. A method according to claim 21 wherein leucovorin at a dosage of about 400 mg/m², 5-fluorouracil (5-FU) at a dosage of about 2800 mg/m², irinotecan at a dosage of about 180 mg/m² and the polypeptide of SEQ ID NO:1, or biosimilar thereof, at a dosage of about 4 mg/kg are administered to patient.

30. A method according to claim 21 wherein leucovorin is administered intravenously at a dosage of about 400 mg/m², 5-fluorouracil (5-FU) is administered intravenously at a dosage of about 2800 mg/m², irinotecan is administered intravenously at a dosage of about 180 mg/m² and the polypeptide of SEQ ID NO:1, or biosimilar thereof, is administered intravenously at a dosage of about 4 mg/kg and wherein the combination is administered every two weeks.

31. A method according to claim 21 wherein the leucovorin, 5-fluorouracil (5-FU), irinotecan and the polypeptide of SEQ ID NO:1, or biosimilar thereof, are administered intravenously every two weeks for a period comprised between 9 and 18 weeks.

32. A method according to claim 21 wherein the leucovorin is administered intravenously immediately after the polypeptide or biosimilar administration.

33. A method according to claim 21 wherein the leucovorin is administered intravenously immediately after the polypeptide or biosimilar administration over a period of about 2 hours.

34. A method according to claim 21 wherein the irinotecan is administered intravenously immediately after the polypeptide or biosimilar administration.

35. A method according to claim 21 wherein the irinotecan is administered intravenously immediately after the polypeptide or biosimilar administration over a period of about 90 minutes.

36. A method according to claim 21 wherein the 5-fluorouracil (5-FU) is administered immediately after the polypeptide or biosimilar administration.

37. A method according to claim 21 wherein a first quantity of 5-fluorouracil (5-FU) is administered intravenously immediately after the polypeptide or biosimilar administration and a second quantity of 5-FU is administered intravenously after the first quantity in continuous infusion.

38. A method according to claim 21 wherein about 400 mg/m² of 5-fluorouracil (5-FU) is administered intravenously over a period of 2 to 4 minutes after the polypeptide or biosimilar administration and wherein 2400 mg/m² of 5-FU is administered intravenously over almost 46 hours after the administration of the 400 mg/m² in continuous infusion.

39. A method according to claim 21 wherein the patient has liver metastases.

40. Polypeptide of SEQ ID NO:1, or biosimilar thereof, for use in treating patients with cancer or cancer symptom wherein:
a) the polypeptide, or biosimilar thereof, should not be administered to patients with a with severe haemorrhage, and/or
b) the polypeptide, or biosimilar thereof, should be discontinued in patients who experience gastrointestinal perforation, and/or
c) the polypeptide, or biosimilar thereof, should be discontinued in patients with compromised wound healing.

41. Polypeptide of SEQ ID NO:1, or biosimilar thereof, according to claim 40 wherein the cancer and cancer symptom are respectively Colorectal Cancer (CRC) and Colorectal Cancer (CRC) symptom.

42. Polypeptide of SEQ ID NO:1, according to claim 40 wherein said polypeptide is aflibercept or ziv-aflibercept or a biosimilar thereof.

43. Composition comprising therapeutically effective amounts of a polypeptide of SEQ ID NO:1, or biosimilar thereof, in combination with leucovorin or folinic acid, 5-fluorouracil (5-FU) and irinocetan and comprising a pharmaceutically acceptable carrier for use in treating patients with Colorectal Cancer (CRC) or Colorectal Cancer (CRC) symptom wherein:
a) the polypeptide, or biosimilar thereof, should not be administered to patients with a with severe haemorrhage, and/or
b) the polypeptide, or biosimilar thereof, should be discontinued in patients who experience gastrointestinal perforation, and/or
c) the polypeptide, or biosimilar thereof, should be discontinued in patients with compromised wound healing.

44. A method of managing the risk of hemorrhage, gastrointestinal perforation and compromised wound healing to allow a safe and effective use of a regiment comprising a polypeptide of SEQ ID NO:1, or biosimilar thereof, leucovorin, 5-fluorouracil (5-FU) and irinotecan in the treatment of patients with colorectal cancer (CRC), said method comprising,
a) assessing whether a patient presents severe haemorrhage, and, if not, administering to said patient said regiment;
b) monitoring said patient for signs of gastrointestinal perforation or compromised wound healing; and
c) discontinuing the regiment if said signs appear.


SEQUENCE LISTING (1 sequence)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG